SEQUENCE LISTING (51 sequences)

SEQ ID NO: 1 - protein, 13 aa, Homo sapiens
LILDFLRGGDLFT

SEQ ID NO: 2 - protein, 13 aa, Homo sapiens
LILEYLSGGELFM

SEQ ID NO: 3 - protein, 11 aa, Homo sapiens
RRRLASTNDKG

SEQ ID NO: 4 - protein, 20 aa, Homo sapiens
VSVSETDDYAEIIDEEDTFT

SEQ ID NO: 5 - RNA, 21 nt, Homo sapiens
aagaagcuggacuucagccgu

SEQ ID NO: 6 - RNA, 21 nt, Homo sapiens
aaccuaugggagaggaggaga

SEQ ID NO: 7 - RNA, 19 nt, Homo sapiens
aauuauggaugaaccuaug

SEQ ID NO: 8 - RNA, 19 nt, Homo sapiens
auuauggaugaaccuaugg

SEQ ID NO: 9 - RNA, 19 nt, Homo sapiens
gcuuuaugccaugaaggua

SEQ ID NO: 10 - RNA, 19 nt, Homo sapiens
ggccacacugaaaguucga

SEQ ID NO: 11 - RNA, 19 nt, Homo sapiens
acgugauaucuugguagag

SEQ ID NO: 12 - RNA, 19 nt, Homo sapiens
uaucuugguagagguuaau

SEQ ID NO: 13 - RNA, 19 nt, Homo sapiens
gauuuguuuacacgcuuau

SEQ ID NO: 14 - RNA, 19 nt, Homo sapiens
uuuguuuacacgcuuaucc

SEQ ID NO: 15 - RNA, 19 nt, Homo sapiens
acuugcacuugcuuuagac

SEQ ID NO: 16 - RNA, 19 nt, Homo sapiens
ggucacaucaaguuaacag

SEQ ID NO: 17 - RNA, 19 nt, Homo sapiens
aagagucuauugaccauga

SEQ ID NO: 18 - RNA, 19 nt, Homo sapiens
agagucuauugaccaugaa

SEQ ID NO: 19 - RNA, 19 nt, Homo sapiens
gagucuauugaccaugaaa

SEQ ID NO: 20 - RNA, 19 nt, Homo sapiens
guuaaucgucgaggucaua

SEQ ID NO: 21 - RNA, 19 nt, Homo sapiens
gugcugacugguggucuuu

SEQ ID NO: 22 - RNA, 19 nt, Homo sapiens
agcgaaauccugcaaacag

SEQ ID NO: 23 - RNA, 19 nt, Homo sapiens
auccugcaaacagauuagg

SEQ ID NO: 24 - RNA, 19 nt, Homo sapiens
uccugcaaacagauuaggu

SEQ ID NO: 25 - RNA, 19 nt, Homo sapiens
acgauagacuggaauaaac

SEQ ID NO: 26 - RNA, 19 nt, Homo sapiens
cgauagacuggaauaaacu

SEQ ID NO: 27 - RNA, 19 nt, Homo sapiens
uagacuggaauaaacugua

SEQ ID NO: 28 - RNA, 19 nt, Homo sapiens
cuggaauaaacuguauaga

SEQ ID NO: 29 - RNA, 19 nt, Homo sapiens
gaugaugaaagccaagcua

SEQ ID NO: 30 - RNA, 19 nt, Homo sapiens
ugaugaaagccaagcuaug

SEQ ID NO: 31 - RNA, 19 nt, Homo sapiens
gcauccaaacauuaucacu

SEQ ID NO: 32 - RNA, 19 nt, Homo sapiens
uccaaacauuaucacucua

SEQ ID NO: 33 - RNA, 19 nt, Homo sapiens
acauuaucacucuaaagga

SEQ ID NO: 34 - RNA, 19 nt, Homo sapiens
cauuaucacucuaaaggau

SEQ ID NO: 35 - RNA, 19 nt, Homo sapiens
uuaucacucuaaaggaugu

SEQ ID NO: 36 - RNA, 19 nt, Homo sapiens
ucacucuaaaggauguaua

SEQ ID NO: 37 - RNA, 19 nt, Homo sapiens
uguguauguaguaacagaa

SEQ ID NO: 38 - RNA, 19 nt, Homo sapiens
uguggaugaaucugguaau

SEQ ID NO: 39 - RNA, 19 nt, Homo sapiens
ucugguaauccggaaucua

SEQ ID NO: 40 - RNA, 19 nt, Homo sapiens
aaauggucuucucaugacu

SEQ ID NO: 41 - RNA, 19 nt, Homo sapiens
caaugcuuaccgguuacac

SEQ ID NO: 42 - RNA, 19 nt, Homo sapiens
ccgguuacacuccauuugc

SEQ ID NO: 43 - RNA, 19 nt, Homo sapiens
gagacugacugcugcucuu

SEQ ID NO: 44 - RNA, 19 nt, Homo sapiens
ccaacugccacaauaccaa

SEQ ID NO: 45 - RNA, 19 nt, Homo sapiens
ugcaccacaucuaguaaag

SEQ ID NO: 46 - RNA, 19 nt, Homo sapiens
uucugcuuugaaccguaau

SEQ ID NO: 47 - RNA, 19 nt, Homo sapiens
ccguaaucagucaccaguu

SEQ ID NO: 48 - DNA, 3206 bp, homo sapiens
ctggtgactcgcggcggcggcggcggacggcccagccggagcgcgaggggctcgggggggcgcggcggttcgggtcgcagagccagggaccccaggacccgggaggcggcgcagccggggccgccggaggagcgcgggtgacctggcggcggcgagatgccgctcgcccagctcaaggagccctggccgctcatggagctagtgccgctggacccggagaatggacagacctcaggggaagaagctggacttcagccgtccaaggatgagggcgtcctcaaggagatctccatcacgcaccacgtcaaggctggctctgagaaggctgatccatcccatttcgagctcctcaaggttctgggccagggatcctttggcaaagtcttcctggtgcggaaagtcacccggcctgacagtgggcacctgtatgctatgaaggtgctgaagaaggcaacgctgaaagtacgtgaccgcgtccggaccaagatggagagagacatcctggctgatgtaaatcacccattcgtggtgaagctgcactatgccttccagaccgagggcaagctctatctcattctggacttcctgcgtggtggggacctcttcacccggctctcaaaagaggtgatgttcacggaggaggatgtgaagttttacctggccgagctggctctgggcctggatcacctgcacagcctgggtatcatttacagagacctcaagcctgagaacatccttctggatgaggagggccacatcaaactcactgactttggcctgagcaaagaggccattgaccacgagaagaaggcctattctttctgcgggacagtggagtacatggcccctgaggtcgtcaaccgccagggccactcccatagtgcggactggtggtcctatggggtgttgatgtttgagatgctgacgggctccctgcccttccaggggaaggaccggaaggagaccatgacactgattctgaaggcgaagctaggcatgccccagtttctgagcactgaagcccagagcctcttgcgggccctgttcaagcggaatcctgccaaccggctcggctccggccctgatggggcagaggaaatcaagcggcatgtcttctactccaccattgactggaataagctataccgtcgtgagatcaagccacccttcaagccagcagtggctcagcctgatgacaccttctactttgacaccgagttcacgtcccgcacacccaaggattccccaggcatcccccccagcgctggggcccatcagctgttccggggcttcagcttcgtggccaccggcctgatggaagacgacggcaagcctcgtgccccgcaggcacccctgcactcggtggtacagcaactccatgggaagaacctggtttttagtgacggctacgtggtaaaggagacaattggtgtgggctcctactctgagtgcaagcgctgtgtccacaaggccaccaacatggagtatgctgtcaaggtcattgataagagcaagcgggatccttcagaagagattgagattcttctgcggtatggccagcaccccaacatcatcactctgaaagatgtgtatgatgatggcaaacacgtgtacctggtgacagagctgatgcggggtggggagctgctggacaagatcctgcggcagaagttcttctcagagcgggaggccagctttgtcctgcacaccattggcaaaactgtggagtatctgcactcacagggggttgtgcacagggacctgaagcccagcaacatcctgtatgtggacgagtccgggaatcccgagtgcctgcgcatctgtgactttggttttgccaaacagctgcgggctgagaatgggctcctcatgacaccttgctacacagccaactttgtggcgcctgaggtgctgaagcgccagggctacgatgaaggctgcgacatctggagcctgggcattctgctgtacaccatgctggcaggatatactccatttgccaacggtcccagtgacacaccagaggaaatcctaacccggatcggcagtgggaagtttaccctcagtgggggaaattggaacacagtttcagagacagccaaggacctggtgtccaagatgctacacgtggatccccaccagcgcctcacagctaagcaggttctgcagcatccatgggtcacccagaaagacaagcttccccaaagccagctgtcccaccaggacctacagcttgtgaagggagccatggctgccacgtactccgcactcaacagctccaagcccaccccccagctgaagcccatcgagtcatccatcctggcccagcggcgagtgaggaagttgccatccaccaccctgtgaggcaccagggcattcgggccacagggcggtgctagcttgacagagtcagcatgcttcccagagggagcaggccggaaccacagggccagagggagctggaacccgaggggccggggaagctgccagcccagaacacccctaatgagggtgtgagaagtgccttctccttccccaggatggactcttctcggctcaggctctgctggtggaaagcgattcactgtataaacttttttttatgaaaaaaatggcatcaaccaccatggatttttacaagatccatttgcctttctgggagcagaaacagccattgcggccccaggaggggaactgagtcacgctggggctctctgagactctttagagcagctttgggatcccaccctggggacccccatgattggccacctgtagccatctgcacacacctccgagacagtccagtgtcacctctctcagagcatctggctgtttagcagaactcattctatccccaatcagctccttttccgttctgttctgctgggagttctagaaccacttcctgctacaggaggggtctcatgtcctgctggcttccagcttcaggcaccagcatccaccttggctctgccagtggatcccctgcggtcaggctgggcagccccagagagaggatgtggaaagcactttttggctgacttcatctggggttggcaacaggacagagttcacaggaggccagtgggcgggccatgagggacagggtcttttttcatttcttcctcagctggttactcagggttcatctgtccatggcctttctaataaactgttgagttgaaaaaaaaaaaaaaaaaaaa

SEQ ID NO: 49 - DNA, 2260 bp, homo sapiens
atgccgctggcgcagctggcggacccgtggcagaagatggctgtggagagcccgtccgacagcgctgagaatggacagcaaattatggatgaacctatgggagaggaggagattaacccacaaactgaagaagtcagtatcaaagaaattgcaatcacacatcatgtaaaggaaggacatgaaaaggcagatccttcccagtttgaacttttaaaagtattagggcagggatcatttggaaaggttttcttagttaaaaaaatctcaggctctgatgctaggcagctttatgccatgaaggtattgaagaaggccacactgaaagttcgagaccgagttcggacaaaaatggaacgtgatatcttggtagaggttaatcatccttttattgtcaagttgcattatgcttttcaaactgaagggaagttgtatcttattttggattttctcaggggaggagatttgtttacacgcttatccaaagaggtgatgttcacagaagaagatgtcaaattctacttggctgaacttgcacttgctttagaccatctacatagcctgggaataatttatagagacttaaaaccagaaaatatacttcttgatgaagaaggtcacatcaagttaacagatttcggcctaagtaaagagtctattgaccatgaaaagaaggcatattctttttgtggaactgtggagtatatggctccagaagtagttaatcgtcgaggtcatactcagagtgctgactggtggtcttttggtgtgttaatgtttgaaatgcttactggtacactccctttccaaggaaaagatcgaaaagaaacaatgactatgattcttaaagccaaacttggaatgccacagtttttgagtcctgaagcgcagagtcttttacgaatgcttttcaagcgaaatcctgcaaacagattaggtgcaggaccagatggagttgaagaaattaaaagacattcatttttctcaacgatagactggaataaactgtatagaagagaaattcatccgccatttaaacctgcaacgggcaggcctgaagatacattctattttgatcctgagtttactgcaaaaactcccaaagattcacctggcattccacctagtgctaatgcacatcagctttttcgggggtttagttttgttgctattacctcagatgatgaaagccaagctatgcagacagttggtgtacattcaattgttcagcagttacacaggaacagtattcagtttactgatggatatgaagtaaaagaagatattggagttggctcctactctgtttgcaagagatgtatacataaagctacaaacatggagtttgcagtgaagattattgataaaagcaagagagacccaacagaagaaattgaaattcttcttcgttatggacagcatccaaacattatcactctaaaggatgtatatgatgatggaaagtatgtgtatgtagtaacagaacttatgaaaggaggtgaattgctggataaaattcttagacaaaaatttttctctgaacgagaggccagtgctgtcctgttcactataactaaaaccgttgaatatcttcacgcacaaggggtggttcatcgagacttgaaacctagcaacattctttatgtggatgaatctggtaatccggaatctattcgaatttgtgattttggctttgcaaaacagctgagagcggaaaatggtcttctcatgactccttgttacactgcaaattttgttgcaccagaggttttaaaaagacaaggctatgatgctgcttgtgatatatggagtcttggtgtcctactctatacaatgcttaccggttacactccatttgcaaatggtcctgatgatacaccagaggaaatattggcacgaataggtagcggaaaattctcactcagtggtggttactggaattctgtttcagacacagcaaaggacctggtgtcaaagatgcttcatgtagaccctcatcagagactgactgctgctcttgtgctcagacatccttggatcgtccactgggaccaactgccacaataccaactaaacagacaggatgcaccacatctagtaaagggtgccatggcagctacatattctgctttgaaccgtaatcagtcaccagttttggaaccagtaggccgctctactcttgctcagcggagaggtattaaaaaaatcacctcaacagccctgtgaagtgacctcagtgagatatttggatccatggtgtaaa

SEQ ID NO: 50 - DNA, 3982 bp, homo sapiens
ggcacgaggcggagaaggaggcggagggagcgattgtggccccggccgcggtggccggcgcggcctgccctttgtgaccgcagctcgcgccccacgccccgcgcccatggccgccgtgccgggctccctggccacgcgtgcccgcccgcggacctgagccccgcgcctgggatgccggggatgcgcgtcccccggccctgcggctgctccgggctgggcgcggggcgatggacctgagcatgaagaagttcgccgtgcgcaggttcttctctgtgtacctgcgcaggaagtcgcgctccaagagctccagcctgagccggctcgaggaagaaggtgtcgtgaaggagatagacatcagccatcatgtgaaggagggctttgagaaggcagatccttcccagtttgagctgctgaaggttttaggacaaggatcctatggaaaggtgttcctggtgaggaaggtgaaggggtccgacgctgggcagctctacgccatgaaggtccttaagaaagccaccctaaaagttcgggaccgagtgagatcgaagatggagagagacatcttggcagaagtgaatcaccccttcattgtgaagcttcattatgcctttcagacggaaggaaagctctacctgatcctggacttcctgcggggaggggacctcttcacccggctctccaaagaggtcatgttcacggaggaggatgtcaagttctacctggctgagctggccttggctttagaccatctccacagcctggggatcatctacagagatctgaagcctgagaacatcctcctggatgaagaggggcacattaagatcacagatttcggcctgagtaaggaggccattgaccacgacaagagagcgtactccttctgcgggacgatcgagtacatggcgcccgaggtggtgaaccggcgaggacacacgcagagtgccgactggtggtccttcggcgtgctcatgtttgagatgctcacggggtccctgccgttccaggggaaggacaggaaggagaccatggctctcatcctcaaagccaagctggggatgccgcagttcctcagtggggaggcacagagtttgctgcgagctctcttcaaacggaacccctgcaaccggctgggtgctggcattgacggagtggaggaaattaagcgccatcccttctttgtgaccatagactggaacacgctgtaccggaaggagatcaagccaccgttcaaaccagcagtgggcaggcctgaggacaccttccactttgaccccgagttcacagcgcggacgcccacagactctcctggcgtccccccgagtgcaaacgctcatcacctgtttagaggattcagctttgtggcctcaagcctgatccaggagccctcacagcaagatctgcacaaagtcccagttcacccaatcgtgcagcagttacacgggaacaacatccacttcaccgatggctacgagatcaaggaggacatcggggtgggctcctactcagtgtgcaagcgatgtgtgcataaagccacagacaccgagtatgccgtgaagatcattgataagagcaagagagacccctcggaagagattgagatcctcctgcggtacggccagcacccgaacatcatcaccctcaaggatgtctatgatgatggcaagtttgtgtacctggtaatggagctgatgcgtggtggggagctcctggaccgcatcctccggcagagatacttctcggagcgcgaagccagtgacgtcctgtgcaccatcaccaagaccatggactacctccattcccagggggttgttcatcgagacctgaagccgagtaacatcctgtacagggatgagtcggggagcccagaatccatccgagtctgcgacttcggctttgccaagcagctgcgcgcggggaacgggctgctcatgacaccctgctacacggccaatttcgtggccccggaggtcctgaagcgtcaaggctatgatgcggcgtgtgacatctggagtttggggatcctgttgtacaccatgctggcaggatttaccccttttgcaaatgggccagacgatacccctgaggagattctggcgcggatcggcagtgggaagtatgccctttctgggggaaactgggactcgatatctgacgcagctaaagacgtcgtgtccaagatgctccacgtggaccctcatcagcgcctgacggcgatgcaagtgctcaaacacccgtgggtggtcaacagagagtacctgtccccaaaccagctcagccgacaggacgtgcacctggtgaagggcgcgatggccgccacctactttgctctaaacagaacacctcaggccccgcggctggagcccgtgctgtcgtccaacctggctcagcgcagaggcatgaagagactcacgtccacgcggttgtagcgggtgggaccctggccccagcgtcccctgccagcatcctcgtgggctcacagaccccggcctcggagcccgtctggcacccagagtgaccacaagtccagcagggaggcggcgcccgccctcgccgtgtccgtgttttctttttcagccccggagagggtcctgacctgggggcttctccaagcctcactgcgccagcctccccgcccgctctcttttctcccaagcaaaaccaaatgcgccccttcacctcgcgtgcccgtgcgaggccgggggcttctttcagagcccgcgggtcctctcatacatggcttctgtttctgccgagagatctgttttccaattatgaagccggtcggtttggtcagactcccgacacccacgtcccaggtacccggtgggaaagtggcagtgcgagggcgcagccattggtggttgcagggccccagagggctggggtgacctggcatcccggggctccccacgggctggatgacggggttggcactgtggcgtccaggaggagatgcctggttctgcccaaaataatccaaagagccgtttcctcctcgcccttcagtttttgcctgaggtgctgggtagcccatcctttcctctgtcccagattcaaatgaggagtaagagcccagacgagaggaaggcaggctggatctttgccttgagagctccgtgtcaccaggatggaagggggtgcctctcggaggagcctgtgtccacctccagtctcggctttccccggggggccaagcgcactgggctgccgtctgtccccagctcccgtggccacacagctatctggaggctttgcagggagtcgtgggttctcgcacctgctcagccctgtgtcggcttcctgtgtgctcacctaaagctgtggttttgctgtgttcacttcgatttttctggtctgtggagaaactgtgaattggagaaatggagctctgtggcttcccacccaaaccttctcagtccagctggaggctggagggagacacaggccccacccagcagactgaggggcagaggcacaggtgggagggcagcggagatcagcgtggacaggagcgatgcactttgtagatgctgtggctttgtgttgcgttttgtgtctctgttgcacagatctgttttttcacactgatccgtattcccctgggtgtgcacacagggcgggtgtggggcatttaggccatgctgtgctctacttcattgagtaaaatcgagtgagaggttccgggcagcaggatcgacgcccagtccagccggcagagggaacacacgggtccttcattgtcctgtaagggtgttgaagatgctccctggcggcccccaagcagactagatgggaggaggcgccgctcagcccctcaccctgcatcactgaagagcggcgcctctgcagcaagcagggcttcaggaggtgcccgctggccacagccaggttttccctaagaagatgttattttgttgggttttgttccccctccatctcgattctcgtacccaactaaaaaaaaaaaaaaaaaaaaaaa

SEQ ID NO: 51 - DNA, 2640 bp, homo sapiens
acggttttttttttttttttttttttttttttttttttttttttttttttttttataaaattattagtataaaaggggaaatgctaccattcgctcctcaggacgagccctgggaccgagaaatggaagtgttcagcggcggcggcgcgagcagcggcgaggtaaatggtcttaaaatggttgatgagccaatggaagagggagaagcagattcttgtcatgatgaaggagttgttaaagaaatccctattactcatcatgttaaggaaggctatgagaaagcagatcctgcacagtttgagttgctcaaggttcttggtcaggggtcatttggaaaggtttttcttgttagaaagaagaccggtcctgatgctgggcagctctatgcaatgaaggtgttaaaaaaagcctctttaaaagttcgagacagagttcggacaaagatggagagggatatactggtggaagtaaatcatccatttattgtcaaattgcactatgcctttcagactgaagggaaactgtacttaatactggattttctcaggggaggagatgttttcacaagattatccaaagaggttctgtttacagaggaagatgtgaaattctacctcgcagaactggcccttgctttggatcatctgcaccaattaggaattgtttatagagacctgaagccagaaaacattttgcttgatgaaataggacatatcaaattaacagattttggactcagcaaggagtcagtagatcaagaaaagaaggcttactcattttgtggtacagtagagtatatggctcctgaagtagtaaataggagaggccattcccagagtgctgattggtggtcatatggtgttcttatgtttgaaatgcttactggtactctgccatttcaaggtaaagacagaaatgagaccatgaatatgatattaaaagcaaaacttggaatgcctcaatttcttagtgctgaagcacaaagtcttctaaggatgttattcaaaaggaatccagcaaatagattgggatcagaaggagttgaagaaatcaaaagacatctgttttttgcaaatattgactgggataaattatataaaagagaagttcaacctcctttcaaacctgcttctggaaaaccagatgatactttttgttttgatcctgaatttactgcaaaaacacctaaagattctcccggtttgccagccagtgcaaatgctcatcagctcttcaaaggattcagctttgttgcaacttctattgcagaagaatataaaatcactcctatcacaagtgcaaatgtattaccaattgttcagataaatggaaatgctgcacaatttggtgaagtatatgaattgaaggaggatattggtgttggctcctactctgtttgcaagcgatgcatacatgcaactaccaacatggaatttgcagtgaagatcattgacaaaagtaagcgagacccttcagaagagattgaaatattgatgcgctatggacaacatcccaacattattactttgaaggatgtctttgatgatggtagatatgtttaccttgttacggatttaatgaaaggaggagagttacttgaccgtattctcaaacaaaaatgtttctcggaacgggaggctagtgatatactatatgtaataagtaagacagttgactatcttcattgtcaaggagttgttcatcgtgatcttaaacctagtaatattttatacatggatgaatcagccagtgcagattcaatcaggatatgtgattttgggtttgcaaaacaacttcgaggagaaaatggacttctcttaactccatgctacactgcaaactttgttgcacctgaggttcttatgcaacagggatatgatgctgcttgtgatatctggagtttaggagtccttttttacacaatgttggctggctacactccatttgctaatggccccaatgatactcctgaagagatactgctgcgtataggcaatggaaaattctctttgagtggtggaaactgggacaatatttcagacggagcaaaggatttgctttcccatatgcttcatatggacccacatcagcggtatactgctgaacaaatattaaagcactcatggataactcacagagaccagttgccaaatgatcagccaaagagaaatgatgtgtcacatgttgttaagggagcaatggttgcaacatactctgccctgactcacaagacctttcaaccagtcctagagcctgtagctgcttcaagcttagcccagcgacggagcatgaaaaagcgaacatcaactggcctgtaagatttgtggtgttcctaggccaaactggatgaagatgaaattaaatgtgtggcttttttcctattcttatcaaaggcatcgttgtctgctaaattacttgaatattaagtaatattaaatccccatttttaggggaagtgagatttaaaaaaccattcacaggtccacaatattcatactatgtgtttgcagtagtgttcaagtgtttatttaagcatataattggtgtccaccaggtcctcacaacttctctgcacacaagcttctaaaattcctttcaaataaagttactttaatatttaaaaaaaaaaaaaaaaaaaaa

(19) United States
(12) Patent Application Publication
(10) Pub. No.: US 2007/0049539 A1
(43) Pub. Date: Mar. 1, 2007

(54) Rsk inhibitors and therapeutic uses thereof

(76) Inventors: Jeffrey A. Smith, Earlysville, VA (US); Deborah A. Lannigan-Macara, Charlottesville, VA (US); Celeste E. Poteet-Smith, Earlysville, VA (US); Sidney M. Hecht, Charlottesville, VA (US); Yaming Xu, Charlottesville, VA (US); David L. Brautigan, Charlottesville, VA (US)

Correspondence Address: UNIVERSITY OF VIRGINIA PATENT FOUNDATION, 250 WEST MAIN STREET, SUITE 300, CHARLOTTESVILLE, VA, 22902, US

(21) Appl. No.: 11/524,159

(22) Filed: Sep. 20, 2006

Related U.S. Application Data
(62) Division of application No. 10/517,328, filed on Dec. 9, 2004.
(60) Provisional application No. 60/388,006, filed on Jun. 12, 2002.
(60) Provisional application No. 60/449,553, filed on Feb. 24, 2003.

(51) Int. Cl. A61K 31/7048

(52) U.S. Cl. 514/27

(57) ABSTRACT

The present invention is directed to novel compounds and compositions that have Rsk specific inhibitory activity. In addition, inhibition of Rsk by the present compounds has been discovered to halt the proliferation of cancer cell lines while having little effect on the proliferation rate of normal cells. Therefore, the present invention identifies Rsk as a target for therapeutic intervention in diseased states in which the disease or the symptoms can be ameliorated by inhibition of Rsk catalytic activity.

### RELATED APPLICATION

This application claims priority under 35 USC § 119(e) to U.S. Provisional Application Ser. No. 60/388,006, filed Jun. 12, 2002, and 60/449,553, filed Feb. 24, 2003, the disclosures of which are incorporated herein by reference.

### BACKGROUND

Signal transduction pathways relay information from a variety of different stimuli leading to multiple cellular responses. Consequently, such pathways have attracted a great deal of attention as potential targets for therapeutic intervention. The Mitogen-activated Protein Kinase (MAPK) signaling pathway is one key pathway that transduces a large variety of external signals, leading to cellular responses that include growth, differentiation, inflammation and apoptosis. Accordingly, MAPK is activated by several diverse signals under normal conditions. However, improper regulation of MAPK, including hyperactivity, has been associated with many diseased states. More particularly, improper regulation of the Mitogen-activated Protein Kinase (MAPK) pathway is a distinguishing characteristic in many tumors as well as neurological diseased states such as epilepsy.

p90 Ribosomal S6 Kinase (Rsk) is a serine/threonine kinase that is a downstream component of the Mitogen-activated Protein Kinase (MAPK) signaling pathway. Therefore, unregulated stimulation of the MAPK pathway results in unregulated Rsk catalytic activity. The contribution of upstream components such as Epidermal Growth Factor Receptor (EGFR) and the products of the proto-oncogenes c-src, ras, and raf to activate the MAPK pathway, resulting in physiological responses by the cell that are associated with diseased states, have been well documented. However, the extent to which these physiological responses function through Rsk is unknown.

The paucity of data concerning key biological roles of the Ser/Thr protein kinase Rsk family in somatic cells results primarily from the difficulty in distinguishing Rsk function from those of MAPK itself and of the many other downstream MAPK effectors. This difficulty has arisen because of the lack of any Rsk-specific inhibitors. Accordingly, a Rsk specific inhibitor is highly desirable for use as a tool for investigating Rsk function under normal conditions and under diseased conditions in which regulation of the MAPK signaling pathway has been compromised. The present invention provides a method for screening and identifying Rsk-specific inhibitors, as well as methods for using compositions comprising such inhibitors for the treatment of diseases associated with elevated Rsk activity.

### SUMMARY OF VARIOUS EMBODIMENTS OF THE INVENTION

In accordance with one embodiment of the invention a composition is provided that comprises a Rsk specific inhibitory compound. The composition comprises natural compounds isolated from the plant Forsteronia refracta, or other natural sources, as well as chemically synthesized related compounds that exhibit activity as Rsk specific inhibitors. Inhibition of Rsk by the present compounds has been discovered to halt the proliferation of cancer cell lines while having little effect on the proliferation rate of normal cells. Therefore, the present invention identifies Rsk as a target for therapeutic intervention in diseased states in which the disease or the symptoms can be ameliorated by inhibition of Rsk catalytic activity or Rsk expression. In another embodiment, overexpression of Rsk is used as a diagnostic marker of cancer in individuals.

### BRIEF DESCRIPTION OF THE DRAWINGS

FIG. 1: Molecular structure of SL0101-1, SL0101-2 and SL0101-3

FIG. 2: Inhibitory potency of SL0111-1, SL0101-2 and SL0101-3. The catalytic activity of Rsk in the presence of increasing concentrations of each compound was measured. The IC₅₀ of each compound was determined to be 90 nM for SL0101-1, 580 nM for SL0101-2 and 190 nM for SL0101-3.

FIG. 3: In vitro specificity of Forsteronia refracta extract. The influence of Forsteronia refracta extract on the catalytic activity of several protein kinases was examined. MSK=Mitogen and Stress activated Protein Kinase; PKA=Protein Kinase A; FAK=Focal Adhesion Kinase; and p70 S6K=a kinase closely related to p90 Rsk.

FIG. 4: SL0101-1 inhibits proliferation of transformed cells but not parental cells. Inhibition of Rsk by SL0101-1 halts proliferation of Ha-ras-transformed NIH/3T3 cells but has little effect on the proliferation rate of non-transformed NIH/3T3 cells compared to that observed with vehicle alone. Cells were treated with vehicle, 50 μM SL0101, or 50 μM PD 98059 (PD 98059 is a MEK-specific inhibitor). Proliferation was measured using Promega CellTiter-Glo™ Luminescent cell viability assay.

FIG. 5. The ability of SL0101-1 and kaempferol to inhibit Rsk catalytic activity was measured using kinase assays with an immobilized substrate in the presence of varying concentrations of SL0101-1 or kaempferol. The extent of phosphorylation was determined using phosphospecific antibodies directly labeled with horseradish peroxidase (HRP)-conjugated or phosphospecific antibodies in combination with HRP-conjugated secondary antibodies. All assays measured the initial reaction velocity.

FIGS. 6A & 6B. SL0101-1 inhibits activity of the amino-terminal kinase domain. FIG. 6A: HA-tagged Rsk2 and an HA-tagged truncation mutant containing the Rsk amino-terminal kinase domain (Rsk2 (1-389)) were transfected into baby hamster kidney 21 (BHK21) cells. The HA-tagged proteins were immunoprecipitated from lysates of EGF-stimulated cells. FIG. 6B: HA-tagged proteins (including the Rsk2-AIL mutant, wherein the Rsk2 adenosine interacting loop is substituted with that of p70 S6K) were immunoprecipitated from the lysates of EGF-stimulated BHK21 cells transiently transfected with the indicated HA-tagged constructs. Assays were performed as described in FIG. 5 in the presence of vehicle, 2 μM SL0101-1 or 2 μM Ro 318220 (a non-specific PKC inhibitor).

FIGS. 7A & 7B. SL0101-1 inhibition of cell proliferation is reversible. FIG. 7A: Ha-Ras-transformed cells were treated with vehicle or 50 μM SL0101-1. After 48 hr the medium was replaced and cells previously incubated with vehicle were maintained in vehicle. Cells that had previously been incubated with SL0101-1 were treated with either SL0101-1 or vehicle (washout). Cell viability was measured 48 hr later. FIG. 7B: Determination of siRNA to inhibit cancer cell proliferation. Duplex siRNAs to a sequence in the bluescript plasmid (Control), Rsk1, Rsk2 or Rsk1 and Rsk2 were transfected into MCF-7 cells. Medium was replaced 24 hr post-transfection and the cells incubated for an additional 48 hr prior to measuring cell viability.

FIGS. 8A-8D SL0101-1 inhibits the proliferation of cancer cells but not normal cells. FIG. 8A demonstrates the results of treating MCF-7 and MCF-10A cells with vehicle or 50 μM SL0101-1 or U0126 (a MEK inhibitor). FIG. 8B demonstrates the results of treating LNCaP cells with vehicle or 50 μM SL0101-1 or 50 μM U0126. FIG. 8C demonstrates the results of treating MCF-7 cells with vehicle or 50 μM SL0101-1 in serum-free medium. FIG. 8D is a Western blot that presents data showing that SL0101-1 does not inhibit kinases of the MAPK pathway upstream of Rsk. Cell viability was measured at indicated time points. Proliferation assays were conducted using CellTiter-Glo Luminescent Cell Viability Assay (Promega), performed 44 hrs after treatment for FIGS. 8A &B and at indicated points for FIG. 8C. The data are expressed relative to time 0.

FIGS. 9A & 9B Rsk2 specifically activates ERα- and AR-mediated transcription. MCF-7 or LNCaP cells (see FIGS. 9A and 9B, respectively) were co-transfected with a luciferase reporter and β-galactosidase expression vectors. Additionally, the cells were transfected with either control vector (V) or a vector encoding constitutively active Rsk2 (Rsk2(Y707A)). The cells were treated with either vehicle, 10 nM estradiol or 5 nM R1881 and/or 100 ng/ml EGF. Luciferase and β-galactosidase activity were determined and the luciferase data were divided by the β-galactosidase activity to control for differences in transfection efficiency. The data were normalized so that, in the vector control, the response to vehicle addition was zero and the response to either estradiol or R18181 was 100. The values are +SEM. *P<0.05 and **P<0.01 (Student's t-test) obtained by comparing the response obtained with the vector control with that obtained with Rsk2 (Y707A).

FIG. 10. Purified SL0101-1 specifically inhibits Rsk2 activity in vitro. Vehicle or inhibitor (5 μM) was added to the kinase mix containing 5 nM of the indicated purified kinases. The reaction was allowed to proceed for 30 mins at room temperature and the data were normalized to the kinase activity obtained in the presence of vehicle.

### DETAILED DESCRIPTION OF EMBODIMENTS

Definitions

In describing and claiming the embodiments, the following terminology will be used in accordance with the definitions set forth below.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, an “effective amount” means an amount sufficient to produce a selected effect. For example, an effective amount of an Rsk inhibitor is an amount of the inhibitor sufficient to suppress Rsk activity. Suppression of Rsk activity can be detected through the use of a serine/threonine kinase assay, such as the kinase assay described in Example 3.

As used herein, the terms “complementary” or “complementarity” are used in reference to polynucleotides (i.e., a sequence of nucleotides) related by the Watson & Crick base-pairing rules, i.e. two nucleic acid sequences that are capable of binding to one another in an anti-parallel base paring arrangement. For example, the sequence 5′ A-G-T 3′ is complementary to the sequence 3′ T-C-A 5′. Complementarity may be “partial,” in which some of the nucleic acids' bases are not matched according to the base pairing rules. Or, there may be “complete” or “total” complementarity between the nucleic acids.

The general chemical terms used in the description of the compounds of the present invention have their usual meanings. For example, the term “alkyl” by itself or as part of another substituent means a straight or branched aliphatic chain having the stated number of carbon atoms.

The term “halo” includes bromo, chloro, fluoro, and iodo.

The term “haloalkyl” as used herein refers to an alkyl radical bearing at least one halogen substituent, for example, chloromethyl, fluoroethyl or trifluoromethyl and the like.

The term “C₁-Cₙ alkyl” wherein n is an integer, as used herein, refers to a branched or linear alkyl group having from one to the specified number of carbon atoms. Typically C₁-C₆ alkyl groups include, but are not limited to, methyl, ethyl, n-propyl, iso-propyl, butyl, iso-butyl, sec-butyl, tert-butyl, pentyl, hexyl and the like.

The term “C₂-Cₙ alkenyl” wherein n is an integer, as used herein, represents an olefinically unsaturated branched or linear group having from 2 to the specified number of carbon atoms and at least one double bond. Examples of such groups include, but are not limited to, 1-propenyl, 2-propenyl, 1,3-butadienyl, 1-butenyl, hexenyl, pentenyl, and the like.

The term “C₂-Cₙ alkynyl” wherein n is an integer, refers to an unsaturated branched or linear group having from 2 to the specified number of carbon atoms and at least one triple bond. Examples of such groups include, but are not limited to, 1-propynyl, 2-propynyl, 1-butynyl, 2-butynyl, 1-pentynyl, and the like.

The term “C₁-C₄ alkoxy” as used herein represents a group of the structure —OR wherein O is oxygen and R is C₁-C₄ alkyl. Examples of such groups include, but are not limited to, methoxy, ethoxy, n-propoxy, isopropoxy, t-butoxy, n-pentoxy and n-hexoxy.

As used herein, the term “optionally substituted” refers to zero to four substituents, wherein the substituents are each independently selected.

As used herein the term “aryl” refers to a mono- or bicyclic carbocyclic ring system having one or two aromatic rings including, but not limited to, phenyl, naphthyl, tetrahydronaphthyl, indanyl, indenyl, and the like. Aryl groups (including bicyclic aryl groups) can be unsubstituted or substituted with one, two or three substituents independently selected from lower alkyl, haloalkyl, alkoxy, amino, alkylamino, dialkylamino, hydroxy, halo, and nitro. Substituted aryl includes aryl compounds having one or two C₁-C₆ alkyl, halo or amino substituents. The term (alkyl)aryl refers to any aryl group which is attached to the parent moiety via the alkyl group.

The term “C₃-Cₙ cycloalkyl” wherein n=4-8, represents cyclopropyl, cyclobutyl, cyclopentyl, cyclohexyl, cycloheptyl, and cyclooctyl.

The term “heterocyclic group” refers to a C₃-C₈ cycloalkyl group containing from one to three heteroatoms wherein the heteroatoms are selected from the group consisting of oxygen, sulfur, and nitrogen.

The term “bicyclic” represents either an unsaturated or saturated stable 7- to 12-membered bridged or fused bicyclic carbon ring. The bicyclic ring may be attached at any carbon atom which affords a stable structure. The term includes, but is not limited to, naphthyl, dicyclohexyl, dicyclohexenyl, and the like.

The term “lower alkyl” as used herein refers to branched or straight chain alkyl groups comprising one to eight carbon atoms, including methyl, ethyl, propyl, isopropyl, n-butyl, t-butyl, neopentyl and the like.

The term, “parenteral” means not through the alimentary canal but by some other route such as subcutaneous, intramuscular, intraspinal, or intravenous.

As used herein, the term “treating” includes administering therapy to prevent, cure, or alleviate/prevent the symptoms associated with, a specific disorder, disease, injury or condition. For example treating cancer includes inhibition or complete growth arrest of a tumor, reduction in the number of tumor cells, reduction in tumor size, inhibition of tumor cell infiltration into peripheral organs/tissues, inhibition of metastasis as well as relief, to some extent, of one or more symptoms associated with the disorder. The treatment of cancer also includes the administration of a therapeutic agent that directly decreases the pathology of tumor cells, or renders the tumor cells more susceptible to treatment by other therapeutic agents, e.g., radiation and/or chemotherapy.

The term “neoplastic cells” as used herein relates to cells that constitute an abnormal new growth, i.e. cells that divide to form tissue that serves no physiological function in the host organism. As used herein, the term “tumor” refers to a mass or population of cells that result from excessive cell division and serve no physiological function in the host organism, whether malignant or benign. A “tumor” is further defined as two or more neoplastic cells. “Malignant tumors” are distinguished from benign growths or tumors in that, in addition to uncontrolled cellular proliferation, they will invade surrounding tissues and may additionally metastasize.

As used herein the term “neoplastic disease” relates to any disease that is characterized by the presence of neoplastic cells. Neoplastic diseases include cancer and other diseases characterized by the uncontrolled, abnormal growth of cells. Examples of cancer include but are not limited to, carcinoma, lymphoma, blastoma, sarcoma, and leukemia. More particular examples of such cancers include breast cancer, prostate cancer, colon cancer, squamous cell cancer, small-cell lung cancer, non-small cell lung cancer, ovarian cancer, cervical cancer, gastrointestinal cancer, pancreatic cancer, glioblastoma, liver cancer, bladder cancer, hepatoma, colorectal cancer, uterine cervical cancer, endometrial carcinoma, salivary gland carcinoma, kidney cancer, vulval cancer, thyroid cancer, hepatic carcinoma, skin cancer, melanoma, brain cancer, ovarian cancer, neuroblastoma, myeloma, various types of head and neck cancer, acute lymphoblastic leukemia, acute myeloid leukemia, Ewing sarcoma and peripheral neuroepithelioma. All of the possible cancers listed herein are included in, or may be excluded from, the present invention as individual species.

As used herein the term “anti-tumor agent” relates to agents known in the art that have been demonstrated to have utility for treating neoplastic disease. For example, antitumor agents include, but are not limited to, antibodies, toxins, chemotherapeutics, enzymes, cytokines, radionuclides, photodynamic agents, and angiogenesis inhibitors. Toxins include ricin A chain, mutant Pseudomonas exotoxins, diphtheria toxoid, streptonigrin, boamycin, saporin, gelonin, and pokeweed antiviral protein. Chemotherapeutics include 5-fluorouracil (5-FU), daunorubicin, cisplatinum, bleomycin, melphalan, taxol, tamoxifen, mitomycin-C, and methotrexate as well as any of the compounds described in U.S. Pat. No. 6,372,719 (the disclosure of which is incorporated herein by reference) as being chemotherapeutic agents. Radionuclides include radiometals. Photodynamic agents include porphyrins and their derivatives. Angiogenesis inhibitors are known in the art and include natural and synthetic biomolecules such as paclitaxel, O-(chloroacetyl-carbomyl)fumagillol (“TNP-470” or “AGM 1470”), thrombospondin-1, thrombospondin-2, angiostatin, human chondrocyte-derived inhibitor of angiogenesis (“hCHIAMP”), cartilage-derived angiogenic inhibitor, platelet factor-4, gro-beta, human interferon-inducible protein 10 (“IP10”), interleukin 12, Ro 318220, tricyclodecan-9-yl xanthate (“D609”), irsogladine, 8,9-dihydroxy-7-methyl-benzo[b]quinolizinium bromide (“GPA 1734”), medroxyprogesterone, a combination of heparin and cortisone, glucosidase inhibitors, genistein, thalidomide, diamino-antraquinone, herbimycin, ursolic acid, and oleanolic acid. Anti-tumor therapy includes the administration of an anti-tumor agent or other therapy, such as radiation treatments, that has been reported as being useful for treating cancer.

As used herein, the use of the term “Rsk” is intended to refer generically to all the human Rsk isotypes, including Rsk1, Rsk2, Rsk3 and Rsk4. Rsk1, Rsk2, Rsk3 and Rsk4 are specific human isotypes that have previously been described in the literature. The nucleic acid and protein sequences of these isotypes are found at Genbank accession numbers NM_{—}002953 (for Rsk1, SEQ ID NO: 48), NM_{—}004586 (for Rsk2, SEQ ID NO: 49), NM_{—}021135 (for Rsk3; SEQ ID NO: 50) and NM_{—}014496 (for Rsk4; SEQ ID NO: 51).

As used herein, the term “Rsk specific inhibitor” includes any compound or condition that inhibits Rsk kinase activity (including any or all of the individual Rsk isotypes) without substantially impacting the activity of other kinases. Such inhibitory effects may result from directly or indirectly interfering with the protein's ability to phosphorylate its substrate, or may result from inhibiting the expression (transcription and/or translation) of Rsk.

As used herein, the term “flavonoid” refers to polyphenolic compounds possessing a carbon skeleton having the general structure:

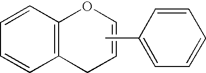

As used herein, the term “SL0101” is used to refer to the three individual compounds, SL0101-1, SL0101-2 and SL0101-3 collectively.

As used herein the term “extract” and like terms refers to a process of separating and/or purifying one or more components from their natural source, or when used as a noun, refers to the composition produced by such a process.

As used herein the term “antisense oligonucleotide” refers to RNA sequences, as well as the DNA sequences encoding for such RNAs, that are complementary to the sequence of a target RNA (or fragment thereof). Typically, the target RNA is a mRNA expressed by a cell.

As used herein the term “interfering oligonucleotide” relates to RNA sequences, as well as the DNA sequences encoding for such RNAs, that are capable of inhibiting the function of a target gene product. More particularly, the interfering oligonucleotide is a polynucleotide sequence that comprises a sequence identical or homologous to a target gene (or fragment thereof). There are two different types of interference RNA (RNAi), short interfering RNA (siRNA) and short hairpin RNA (shRNA). Short interfering RNAs typically consist of 19-22 nt double-stranded RNA molecules that can be chemically synthesized, or generated from larger (>100 nucleotide) double stranded RNA (dsRNA) by enzymatic cleavage using an RNase III-like enzyme called Dicer. Short hairpin RNA, consists of 19-29 nt palindromic sequences connected by loop sequences, that are prepared by chemical synthesis or through recombinant DNA techniques.

### Embodiments

The present invention is directed to compositions comprising a Rsk specific inhibitor and methods of using such compositions for treating disease states related to Rsk hyperactivity. As described herein Rsk-specific inhibitory activity was first identified in a botanical extract through the use of a novel high throughput screening (HTS) Enzyme-Linked Immunosorbent Assay (ELISA) that produces luminescence as a measure of substrate phosphorylation. To discriminate extracts containing Ser/Thr kinase inhibitors from those containing nuisance compounds, a dual screen of the extracts was performed using either a constitutively active mutant of isoform 2 of Rsk (Rsk2) or the catalytic domain of the tyrosine kinase, Focal Adhesion Kinase (FAK). Of 1500 botanical extracts assayed, only one, from Forsteronia refracta inhibited Rsk2 without inhibiting FAK. Forsteronia refracta is a member of the Dogbane family and is native to the South American rain forest. More particularly, the plant is native to Southeastern Brazil, from Goias and Minas Gerais south to Rio Grande do Sul, and in neighboring Misiones, Argentina and Paraguay and is found mostly in upland and riverine forests. Further characteristics of the plant and its availability can be found at http://scisun.nybg.org:8890/searchdb/owa/wwwspecimen.searchform.

To determine whether the Forsteronia refracta extract contained a general Ser/Thr kinase inhibitor, activities of the archetypal Ser/Thr kinase, protein kinase A (PKA) and of two kinases most closely related to Rsk2, p70 S6K and Msk1, were measured in the presence of varying amounts of extract (FIG. 3). Amounts of extract that inhibited Rsk2 activity by 90% did not inhibit PKA, p70 S6K or Msk1 to a greater extent than FAK. Thus, the F. refracta extract contains an inhibitor with remarkable specificity for Rsk2 relative to these other AGC kinase family members.

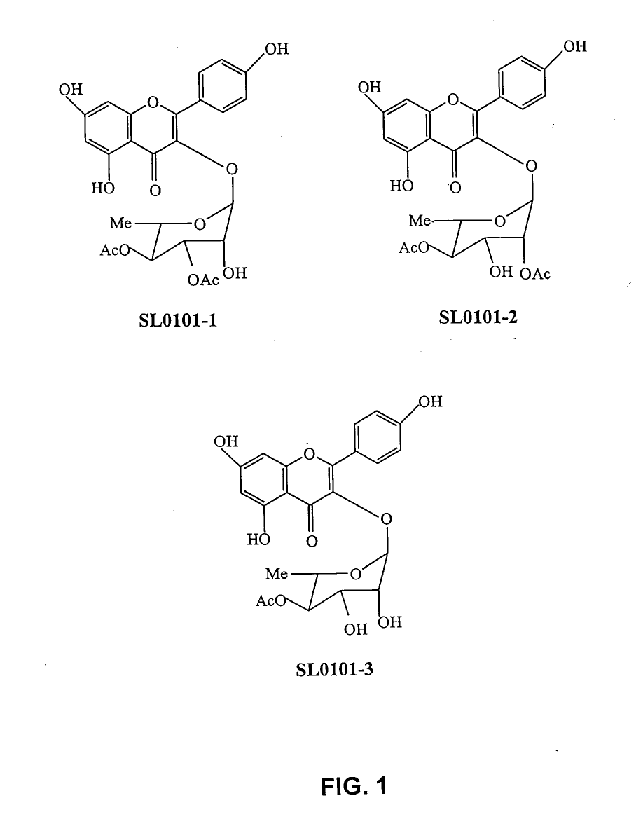

In accordance with one embodiment of the present invention a composition is provided comprising an extract from the plant Forsteronia refracta (a member of the dogbane family found in the South American rain forest) wherein the extract has activity as a Rsk specific inhibitor. In one embodiment the wood stem and/or stem bark of Forsteronia refracta are extracted with an aqueous solvent to purify flavonoid compounds that have Rsk specific inhibitory activity. More particularly, the present invention is directed to a composition comprising an alcohol (e.g. methanol) extract of wood stem and/or stem bark of Forsteronia refracta, or a derivative product thereof, that contains Rsk specific inhibitory activity. The original alcohol extract can be dried to form a powder, or dried and resuspended, or otherwise reconstituted to prepare a non-alcohol solvent based extract comprising the Rsk specific inhibitory compounds of the present invention. In one embodiment the present invention is directed to an extract of Forsteronia refracta tissues, wherein the extract comprises one or more of the flavonoids shown in FIG. 1. In one embodiment the extract is enriched, relative to other components present in the natural tissues, for flavonoid compounds having Rsk specific inhibitory activity. In other words the flavonoid compounds are present in a higher concentration in the extract relative to their concentration in the natural tissues. In one embodiment the extract represents a composition comprising purified flavonoid compounds of F. refracta.

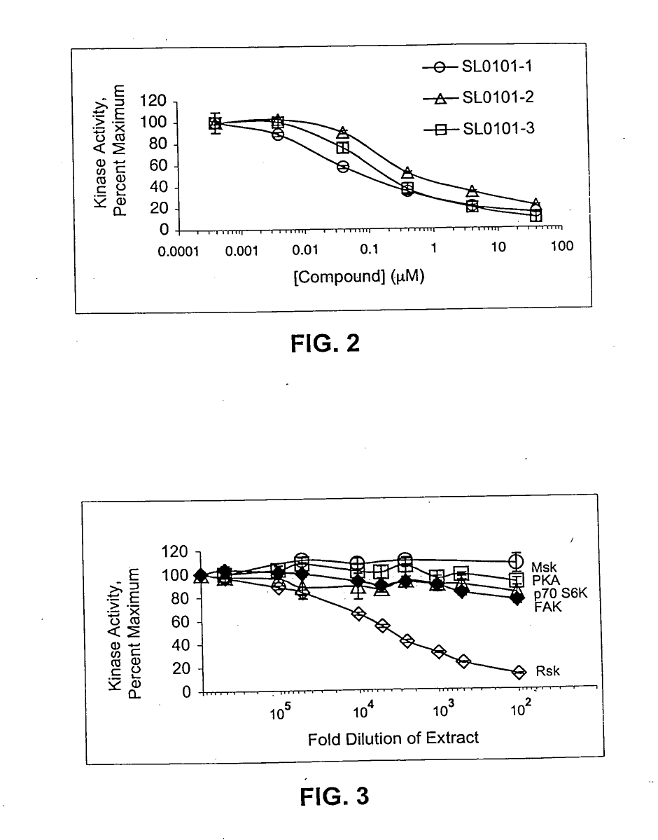

The effect of the F. refracta extract of the present invention on Rsk2 activity was measured in the presence of increasing concentrations of ATP. The addition of the extract did not reduce the maximal velocity of the reaction, but increased the concentration of ATP required to support half-maximal velocity by approximately 20-fold. Thus, the mechanism of Rsk inhibition by the extract is competitive with respect to ATP and SL0101-1, SL0101-2 and SL0101-3 are likely ATP-mimetics. These three compounds have been found inhibit Rsk in vitro with IC₅₀ values of 90 nM, 580 nM and 190 nM, respectively (see FIG. 2). Significantly, these three inhibitors do not inhibit the evolutionarily related p70 S6 kinase and Mitogen and Stress-activated Protein Kinase (MSK). In addition, they do not inhibit the prototypical serine/threonine kinase Protein Kinase A or the tyrosine kinase Focal Adhesion Kinase (FAK) (FIG. 3).

In accordance with one embodiment, the present invention is directed to compounds represented by the general structure:

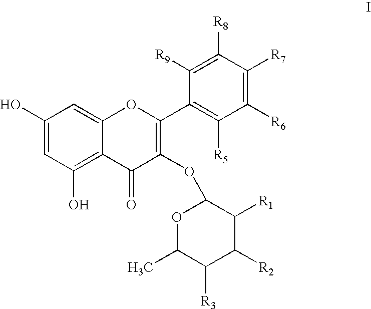

wherein R₁, R₂, and R₃, are independently selected from the group consisting of hydroxy —OCOR₄, —COR₄, C₁-C₄ alkoxy, —O-glucoside and —O-rhamnoside, R₅, R₆, R₇, R₈ and R₉ are independently selected from the group consisting of H, hydroxy —OCOR₄, —COR₄, C₁-C₄ alkoxy, —O-glucoside and —O-rhamnoside, and R₄ is H or C₁-C₄ alkyl, with the proviso that R₁, R₂ and R₃ are not all hydroxy. One embodiment of the invention is directed to a compound of Formula I, wherein R₁, R₂ and R₃ are independently selected from the group consisting of hydroxy and —OCOR₄, R₅ and R₉ are each H, R₆, R₇, and R₈ are independently selected from the group consisting of H, —OR₄, —OCOR₄, and —COR₄ and R₄ is H or methyl, with the proviso that R₁, R₂ and R₃ are not all hydroxy. In one embodiment R₁ and R₂ are independently selected from the group consisting of hydroxy, —COR₄, C₁-C₄ alkoxy and —OCOCH₃, R₃ is —OCOCH₃, R₄ is H or methyl, R₅, R₈ and R₉ are each H, R₆ is H or hydroxy, and R₇ is hydroxy. In an alternative embodiment R₁ and R₂ are independently selected from the group consisting of hydroxy and —OCOCH₃, R₃ is —OCOCH₃, R₅, R₈ and R₉ are each H, R₆ is H or hydroxy, and R₇ is hydroxy.

In one embodiment a compound is provided represented by the general structure of Formula I wherein R₁, R₂ and R₃ are independently selected from the group consisting of hydroxy —OCOR₄, —COR₄, C₁-C₄ alkoxy, —O-glucoside and —O-rhamnoside, R₄ is H or C₁-C₄ alkyl, R₅, R₈ and R₉ are independently selected from the group consisting of H, hydroxy —OCOR₄, —COR₄ and C₁-C₄ alkoxy and R₆ and R₇ are independently selected from the group consisting of hydroxy —OCOR₄, —COR₄ and C₁-C₄ alkoxy. In one embodiment a compound of Formula I is provided wherein R₁, R₂ and R₃ are independently selected from the group consisting of hydroxy and —OCOR₄, R₄ is H or methyl, R₅ and R₉ are each H, R₆ and R₇ are independently selected from the group consisting of hydroxy —OCOR₄, —COR₄ and C₁-C₄ alkoxy, and R₉ is selected from the group consisting of H, —OR₄, —OCOR₄, and —COR₄ and C₁-C₄ alkoxy. In another embodiment a compound of Formula I is provided wherein R₁ and R₂ are independently selected from the group consisting of hydroxy and —OCOCH₃, R₃ is —OCOCH₃, R₅, R₉ and R₉ are each H and R₆ and R₇ are both hydroxy

In one embodiment the present invention is directed to a compound represented by the general structure:

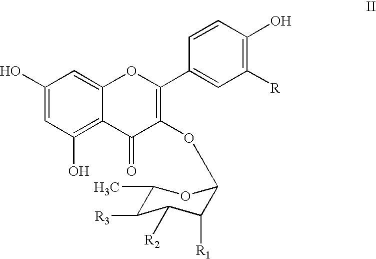

wherein R is H or OH, and R₁, R₂ and R₃ are independently selected from the group consisting of hydroxy —OCOR₄, —COR₄, C₁-C₄ alkoxy, —O-glucoside and —O-rhamnoside, and R₄ is H or —CH₃, with the proviso that R₁, R₂ and R₃ are not all hydroxy. In one embodiment R is H or OH and R₁ and R₂ are independently selected from the group consisting of hydroxy and —OCOCH₃ and R₃ is —OCOCH₃. In another embodiment R is H and R₁, R₂ and R₃ are independently selected from the group consisting of hydroxy —OCOCH₃, —COCH₃, C₁-C₄ alkoxy, —O-glucoside and —O-rhamnoside. In one embodiment R is H and R₁, R₂ and R₃ are independently selected from the group consisting of hydroxy and —OCOCH₃. In one embodiment the compound has the general structure of Formula II wherein R is H, R₁ and R₂ are independently selected from the group consisting of hydroxy and —OCOCH₃ and R₃ is —OCOCH₃. More particularly, in one embodiment a composition is provided comprising one or more compounds having the general structure of Formula II wherein R is H, R₁ is hydroxy and R₂ and R₃ are each 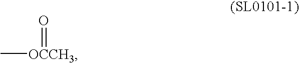
or R is H, R₂ is hydroxy and R₁ and R₃ are each 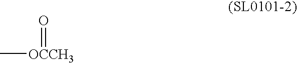
or R is H, R₁ and R₂ are hydroxy and R₃ is 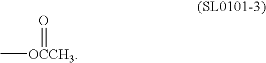
The individual compounds, SL0101-1, SL0101-2 and SL0101-3 are collectively referred to as SL0101.

The purified flavonoid compounds and Rsk specific inhibitory extracts of the present invention can be combined with pharmaceutically acceptable carriers, stabilizing agents, solubilizing agents, and fillers known to those skilled in the art to prepare pharmaceuticals for administration to warm blooded vertebrates. The compositions can be formulated using standard delivery vehicles and standard formulations for oral, parenteral, inhalation or transdermal delivery. Such pharmaceuticals have use in treating neoplastic disease, neurological diseased states (such as epilepsy) or other disease states characterized by inappropriate Rsk activity.

In accordance with one embodiment of the invention a Rsk specific inhibitory compound or composition (i.e. a Rsk specific inhibitory extract, or specific flavonoid compound), is combined with one or more antitumor agents, including those selected from the group consisting of antibodies, toxins, chemotherapeutics, enzymes, cytokines, radionuclides, photodynamic agents, and angiogenesis inhibitors to prepare a pharmaceutical composition. Toxins include ricin A chain, mutant Pseudomonas exotoxins, diphtheria toxoid, streptonigrin, boamycin, saporin, gelonin, and pokeweed antiviral protein. Chemotherapeutics include 5-fluorouracil (5-FU), daunorubicin, cisplatinum, bleomycin, melphalan, taxol, tamoxifen, mitomycin-C, and methotrexate. Radionuclides include radiometals. Photodynamic agents include porphyrins and their derivatives. Angiogenesis inhibitors are known in the art and include natural and synthetic biomolecules such as paclitaxel, O-(chloroacetyl-carbomyl)fumagillol (“TNP-470” or “AGM 1470”), thrombospondin-1, thrombospondin-2, angiostatin, human chondrocyte-derived inhibitor of angiogenesis (“hCHIAMP”), cartilage-derived angiogenic inhibitor, platelet factor-4, gro-beta, human interferon-inducible protein 10 (“IP10”), interleukin 12, Ro 318220, tricyclodecan-9-yl xanthate (“D609”), irsogladine, 8,9-dihydroxy-7-methyl-benzo[b]quinolizinium bromide (“GPA 1734”), medroxyprogesterone, a combination of heparin and cortisone, glucosidase inhibitors, genistein, thalidomide, diamino-antraquinone, herbimycin, ursolic acid, and oleanolic acid.

In accordance with one embodiment, the Rsk specific inhibitor comprises an extract from Forsteronia refracta or from the rhizomes of Zingiber zerumbet. In another embodiment the Rsk specific inhibitor is an interference RNA or a compound of the general structure of Formula I. In accordance with one embodiment of the present invention a composition is provided comprising a chemotherapeutic agent and a compound represented by the general formula

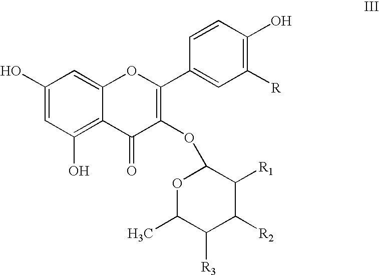

wherein R is H or OH, and R₁, R₂ and R₃ are independently selected from the group consisting of hydroxy —OCOR₄, —COR₄, C₁-C₄ alkoxy, —O-glucoside and —O-rhamnoside, and R₄ is H or —CH₃. In one embodiment the composition comprises a chemotherapeutic agent and the compound of Formula II or III wherein R is H or OH, R₁ and R₂ are independently selected from the group consisting of hydroxy and —OCOCH₃ and R₃ is —OCOCH₃.

One aspect of the present invention is directed to a method of preparing a F. refracta extract that exhibits Rsk specific inhibitory activity, wherein the extract is prepared by extracting tissues, selected from the group consisting of wood stem and stem bark of Forsteronia refracta, with an alcohol solution. In accordance with one embodiment, the wood stem and/or bark of F. refracta is contacted with an alcohol (such as methanol), or an alcohol (methanol) containing solution, for a predetermined length of time at room temperature (about 20° to 25° C.) with or without agitation. The length of time for soaking the plant material can be varied; the tissue simply should be soaked long enough to extract the organic materials in the sample. This can be checked by using fresh solvent samples until negligible or no further flavonoid compounds are extracted. Whether or not additional flavonoid compounds are being extracted with fresh rounds of solvent can be determined either visually or spectroscopically (flavanoids are typically pigmented compounds) or by analytical techniques such as NMR or mass spectrometer. In accordance with one embodiment the tissue can be chopped, shredded, ground or macerated/crushed prior to being treated with an aqueous solvent. In one embodiment, the tissue is sequentially soaked in separate fresh solutions of methanol at room temperature, followed by combination of the methanol solutions and concentration of the solution under diminished pressure to afford a crude extract. In one embodiment the tissue is sequentially soaked in three separate fresh solutions of methanol and the methanol solutions are subsequently combined and concentrated.

The extracted compounds can then be purified using standard techniques. For example, the crude extract material can be applied to a polyamide 6S column (such as a 40-g polyamide 6S column) and washed successively with H₂O, 1:1H₂O—MeOH, 9:1 CH₂Cl₂—MeOH, 1:1 CH₂Cl₂—MeOH and 9:1 MeOH—NH₄OH to afford five fractions. The volume of the washes can be varied, and in one embodiment the column is washed successively with 150 mL of each solvent. The 1:1 CH₂Cl₂—MeOH fraction is then recovered and potentially further fractionated on a diol gel column (such as a 30-g diol gel column).

Extracts of the rhizomes of Zingiber zerumbet can be prepared using techniques described in the prior art. For example, fresh rhizomes of Zingiber zerumbet are crushed and extracted (three to four times) with Me₂CO at room temperature. After filtration, the Me₂CO is evaporated. The residual H₂O solution is then extracted with hexane, CH₂Cl₂ and EtOAc, successively. The EtOAc layer is concentrated and subjected to Sephadex LH-20 CC, silica gel CC (10-20% MeOH in CHCl₃, 1% HOAc in EtOAc) and HPLC (column: Develosil ODS-10/20, solv.: 60% MeOH in H₂O, flow rate: 10 ml min⁻¹) to provide an extract comprising a Rsk inhibitory compound.

In accordance with one embodiment a composition comprising a Rsk inhibitor is prepared from Forsteronia refracta by extracting the tissues as described above. In one embodiment the wood stem and/or stem bark of Forsteronia refracta is extracted with an alcohol solution, such as methanol and the extracted material is applied to a polyamide 6S column. The 6S column is washed successively with H₂O, 1:1H₂O—MeOH, 9:1 CH₂Cl₂—MeOH and 1:1 CH₂Cl₂—MeOH to afford four separate fractions, and the 1:1 CH₂Cl₂—MeOH fraction is recovered. This fraction is then applied to a diol gel column and washed successively with CH₂Cl₂, 99:1 CH₂Cl₂—MeOH and 95:5 CH₂Cl₂—MeOH, 90:10 CH₂Cl₂—MeOH and MeOH. The 95:5 CH₂Cl₂—MeOH fraction and the 90:10 CH₂Cl₂—MeOH fraction are recovered as the fractions comprising Rsk inhibitory activity. Each of these fractions can be subjected to further purification steps such as a C₁₈ reverse phase HPLC column with elution using 65:35 MeOH—H₂O or similar solvent.

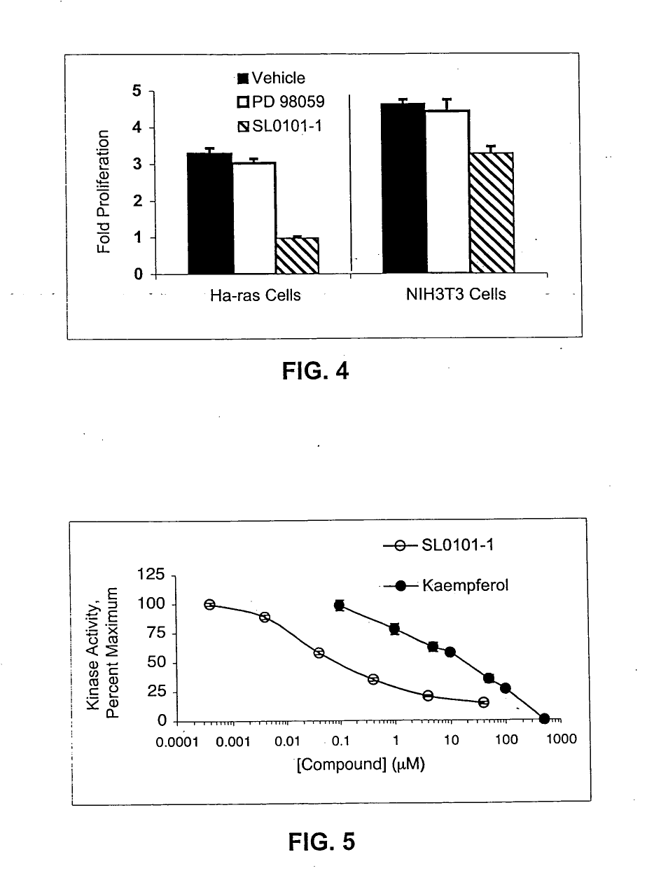

The Rsk-specific inhibitors of the present invention have been shown to inhibit proliferation of a transformed cell without substantially altering the proliferation rate of non-transformed cell growth. Therefore, the inhibitors of the present invention are not toxic to non-transformed cells. For example, as detailed in Example 4, the specific inhibition of Rsk inhibits proliferation of Ha-ras-transformed NIH/3T3 cells without influencing the proliferation rate of non-transformed NIH/3T3 cells. Ha-ras-transformed NIH/3T3 cells or parental NIH/3T3 cells were incubated in the presence of vehicle, 50 μM SL0101-1, or 50 μM PD 98059, a MEK-specific inhibitor. The presence of SL0101-1 inhibits Ha-ras-transformed NIH/3T3 cell proliferation over a 48 hour time course, even in the presence of 10% fetal calf serum (FIG. 4). However, SL0101-1 had little influence on the rate of parental NIH/3T3 proliferation compared to that observed in the presence of vehicle. An influence on the proliferation rate by the MEK inhibitor, PD 98059 was observed only when cells were incubated in the presence of low concentrations of fetal calf serum (0.1-1%).

These data suggest that Rsk-specific inhibitors abolish the growth of malignant tumors without toxic effects on the normal tissues, and thus such compounds can be used as anti-cancer therapeutics. In accordance with one embodiment of the present invention a method of inhibiting or reducing cell proliferation in a human or mammal in need of such treatment is provided. The method comprises the steps of administering to a patient in need thereof a composition comprising a Rsk specific inhibitor, wherein the inhibitor is selected from the group of small-molecules, interference RNA, antisense RNA, antibodies and purified natural products comprising flavonoid compounds represented by the general structure of Formula I.

Small-Molecule Inhibitors

In one embodiment a method is provided for inhibiting the growth of neoplastic cells. The method comprises the steps of administering to the human or mammalian patient a Rsk specific inhibitory composition in an amount effective to decrease or inhibit Rsk activity in the target cells. In one embodiment the Rsk specific inhibitory composition comprises a compound represented by the general structure:

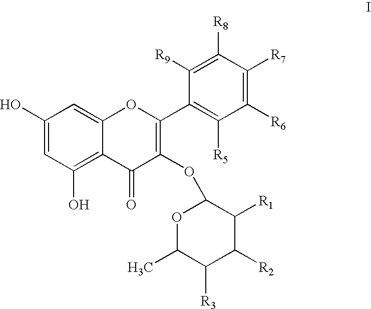

wherein R₁, R₂, and R₃, are independently selected from the group consisting of hydroxy —OCOR₄, —COR₄, C₁-C₄ alkoxy, —O-glucoside and —O-rhamnoside, R₅, R₆, R₇, R₈ and R₉ are independently selected from the group consisting of H, hydroxy —OCOR₄, —COR₄, C₁-C₄ alkoxy, —O-glucoside and —O-rhamnoside, and R₄ is H or C₁-C₄ alkyl. One embodiment of the invention the method comprises administering a compound of Formula I, wherein R₁, R₂ and R₃ are independently selected from the group consisting of hydroxy and —OCOR₄, R₅ and R₉ are each H, R₆, R₇, and R₈ are independently selected from the group consisting of H, —OR₄, —OCOR₄, and —COR₄ and R₄ is H or methyl. In another embodiment R₁ and R₂ are independently selected from the group consisting of hydroxy, —COR₄, C₁-C₄ alkoxy and —OCOCH₃, R₃ is —OCOCH₃, R₄ is H or methyl, R₅, R₈ and R₉ are each H, R₆ is H or hydroxy, and R₇ is hydroxy. In an alternative embodiment R₁ and R₂ are independently selected from the group consisting of hydroxy and —OCOCH₃, R₃ is —OCOCH₃, R₅, R₈ and R₉ are each H, R₆ is H or hydroxy, and R₇ is hydroxy. In one embodiment R₁, R₂ and R₃ are independently selected from the group consisting of hydroxy, —OCOCH₃, —COCH₃, C₁-C₄ alkoxy, —O-glucoside and —O-rhamnoside, R₅, R₆, R₈ and R₉ are each H and R₇ is hydroxy. In another embodiment R₁ and R₂ are independently selected from the group consisting of hydroxy and —OCOCH₃, R₃ is —OCOCH₃, R₅, R₆, R₈ and R₉ are each H and R₇ is hydroxy.

In an alternative embodiment the Rsk specific inhibitory composition comprises an extract of Forsteronia refracta or Zingiber zerumbet. In one embodiment the Rsk specific inhibitory composition comprises a compound represented by the general structure:

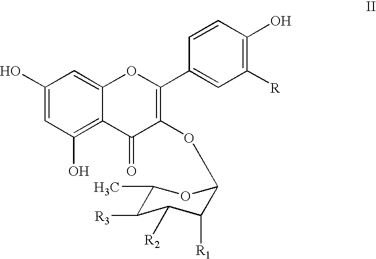

wherein R is H or OH, and R₁, R₂ and R₃ are independently selected from the group consisting of hydroxy, —OCOR₄, —COR₄, C₁-C₄ alkoxy, —O-glucoside and —O-rhamnoside, and R₄ is H or C₁-C₄ alkyl. In one embodiment the composition comprises a compound of Formula II wherein R is H, R₃ is —OCOCH₃ and R₁ and R₂ are independently selected from the group consisting of hydroxy and —OCOCH₃.

Interference and Antisense RNA Inhibitors

In another embodiment a method is provided for inhibiting the growth of neoplastic cells through the use of oligonucleotide agents. In this embodiment the method comprises the steps of administering to a patient a Rsk specific inhibitory composition comprising an anti-sense oligonucleotide or interfering oligonucleotide directed against Rsk1, Rsk2, Rsk3 or Rsk4. The ability to specifically inhibit gene function in a variety of organisms utilizing antisense RNA or ds RNA-mediated interference is well known in the fields of molecular biology (see for example C. P. Hunter, Current Biology [1999] 9:R440-442; Hamilton et al., [1999] Science, 286:950-952; and S. W. Ding, Current Opinions in Biotechnology [2000] 11:152-156, hereby incorporated by reference in their entireties).

Interfering oligonucleotides include RNA interference molecules (RNAi)s as well as the DNA sequences encoding for such RNAi. RNAi in mammalian systems includes the presence of short interfering RNA (siRNA) or short hairpin RNA (shRNA). siRNA typically consists of 19-22 nt double-stranded RNA molecules, whereas shRNA typically consists of 19-29 nt palindromic sequences connected by loop sequences, that mimic the structures of micro RNAi. However, larger or smaller nucleic acid sequences than the ranges cited above can be used for the siRNA and shRNA constructs. Down regulation of gene expression is believed to be achieved in a sequence-specific manner by pairing between homologous RNAi sequences and the target RNA. In one embodiment an siRNA construct is prepared comprising a dsRNA that further comprises a 3′ two nucleotide overhang off the sense and antisence strands as described in Elbashir and Tuschl (2001). Genes & Dev. 15: 188-200.

siRNA and shRNA can be introduced into target cells using standard nucleic acid constructs and techniques known to those skilled in the art. For example, a stable system for expressing siRNA or shRNA has been previously described and utilized to generate transgenic animals (Hasuwa et al. FEBS Lett 532, 227-30 (2002), Rubinson et al. Nat Genet 33, 401-6 (2003) and Carmell et al. Nat Struct Biol 10, 91-2 (2003)). Furthermore, numerous resources are available that describe the design and optimization of RNAi constructs and their use. For example see U.S. Pat. No. 6,506,559 (the disclosure of which is incorporated herein by reference), or the Tushl lab (Rockerfeller University) website for the “siRNA user guide”, located at http://www.mpibpc.gwdg.de/abteilungen/100/105/sirna.html, or information provided by Ambion, Inc. (2130 Woodward, Austin, Tex. 78744-1832, USA), Sirna Thrapeutics (2950 Wilderness Place, Boulder, Colo. 80301), or RNA-TEC NV (Provisorium 2, Minderbroedersstraat 17-19, B-3000 Leuven, Belgium). Expression Cassettes Kits for expression of RNAi sequences in cells are commercially available from Ambion, Inc.

Advantageously, the use of antisense and interference RNAs should allow for the design of antisense or interference RNAs that are specific for the individual Rsk isotypes. Since the various Rsk isotypes demonstrate differences in tissue distribution and embryonic expression there may be therapeutic advantages to inhibiting one Rsk isotype verses another. For example, human Rsk1 is strongly expressed in lymphocytes and is also present in muscle, liver and adipose tissue, whereas human Rsk2 is strongly expressed in fibroblasts and is also present in muscle and placenta lymphocytes with negligible expression in the liver and adipose. Human Rsk3 appears to be expressed ubiquitously. Programs are publicly available for selecting siRNA sequences from a known target gene sequence and thus the design of isotype specific RNAi sequences are well within the skill of the ordinary practitioner once the target sequence is identified. For example, see Dharmacon, Inc., 1376 Miners Drive #101, Lafayette, Colo. 80026, at the Dharmacon siDESIGN Center: http://www.dharmacon.com/. In one embodiment the interfering RNA (RNAi) construct comprises a nucleic acid sequence selected from the group consisting of

| | |
|---|---|
| AAGAAGCUGGACUUCAGCCGU | (SEQ ID NO: 5) |
| AACCUAUGGGAGAGGAGGAGA | (SEQ ID NO: 6) |
| AAUUAUGGAUGAACCUAUG | (SEQ ID NO: 7) |
| AUUAUGGAUGAACCUAUGG | (SEQ ID NO: 8) |
| GCUUUAUGCCAUGAAGGUA | (SEQ ID NO: 9) |
| GGCCACACUGAAAGUUCGA | (SEQ ID NO: 10) |
| ACGUGAUAUCUUGGUAGAG | (SEQ ID NO: 11) |
| UAUCUUGGUAGAGGUUAAU | (SEQ ID NO: 12) |
| GAUUUGUUUACACGCUUAU | (SEQ ID NO: 13) |
| UUUGUUUACACGCUUAUCC | (SEQ ID NO: 14) |
| ACUUGCACUUGCUUUAGAC | (SEQ ID NO: 15) |
| GGUCACAUCAAGUUAACAG | (SEQ ID NO: 16) |
| AAGAGUCUAUUGACCAUGA | (SEQ ID NO: 17) |
| AGAGUCUAUUGACCAUGAA | (SEQ ID NO: 18) |
| GAGUCUAUUGACCAUGAAA | (SEQ ID NO: 19) |
| GUUAAUCGUCGAGGUCAUA | (SEQ ID NO: 20) |
| GUGCUGACUGGUGGUCUUU | (SEQ ID NO: 21) |
| AGCGAAAUCCUGCAAACAG | (SEQ ID NO: 22) |
| AUCCUGCAAACAGAUUAGG | (SEQ ID NO: 23) |
| UCCUGCAAACAGAUUAGGU | (SEQ ID NO: 24) |
| ACGAUAGACUGGAAUAAAC | (SEQ ID NO: 25) |
| CGAUAGACUGGAAUAAACU | (SEQ ID NO: 26) |
| UAGACUGGAAUAAACUGUA | (SEQ ID NO: 27) |
| CUGGAAUAAACUGUAUAGA | (SEQ ID NO: 28) |
| GAUGAUGAAAGCCAAGCUA | (SEQ ID NO: 29) |
| UGAUGAAAGCCAAGCUAUG | (SEQ ID NO: 30) |
| GCAUCCAAACAUUAUCACU | (SEQ ID NO: 31) |
| UCCAAACAUUAUCACUCUA | (SEQ ID NO: 32) |
| ACAUUAUCACUCUAAAGGA | (SEQ ID NO: 33) |
| CAUUAUCACUCUAAAGGAU | (SEQ ID NO: 34) |
| UUAUCACUCUAAAGGAUGU | (SEQ ID NO: 35) |
| UCACUCUAAAGGAUGUAUA | (SEQ ID NO: 36) |
| UGUGUAUGUAGUAACAGAA | (SEQ ID NO: 37) |
| UGUGGAUGAAUCUGGUAAU | (SEQ ID NO: 38) |
| UCUGGUAAUCCGGAAUCUA | (SEQ ID NO: 39) |
| AAAUGGUCUUCUCAUGACU | (SEQ ID NO: 40) |
| CAAUGCUUACCGGUUACAC | (SEQ ID NO: 41) |
| CCGGUUACACUCCAUUUGC | (SEQ ID NO: 42) |
| GAGACUGACUGCUGCUCUU | (SEQ ID NO: 43) |
| CCAACUGCCACAAUACCAA | (SEQ ID NO: 44) |
| UGCACCACAUCUAGUAAAG | (SEQ ID NO: 45) |
| UUCUGCUUUGAACCGUAAU | (SEQ ID NO: 46) |
| CCGUAAUCAGUCACCAGUU | (SEQ ID NO: 47) |

or alternatively, the DNA equivalents of SEQ ID NO: 5-47. In one embodiment the RNAi comprises a sequence selected from the group consisting of SEQ ID NO: 5-47 linked to its complementary sequence either by a covalent linkage or simply by hydrogen bonding. In one embodiment the RNAi comprises a sense and anti-sense RNA sequences that are covalently bound to one another by a linking sequence. The linking sequence is non-complementary to either of the adjoining sequences, thus allowing the formation a stem loop structure upon hybridization of a sequence of SEQ ID NO: 5-47 to its complementary sequence.

Antibody-Based Inhibitors

In another embodiment a method is provided for inhibiting the growth of neoplastic cells through the use of antibodies that are specific for Rsk. In accordance with one embodiment the Rsk specific inhibitory composition comprises an antibody that is specific for Rsk, and in one embodiment the antibody is specific for a Rsk isotype selected from the group consisting of Rsk1, Rsk2, Rsk3 and Rsk4. In accordance with one embodiment the Rsk specific antibody is directed against the adenosine interacting loop of a Rsk enzyme, including for example, the AIL of a Rsk-isotype selected from the group consisting of Rsk1, Rsk2, Rsk3 and Rsk4. Antibodies suitable for use as Rsk specific inhibitory compounds include both monoclonal and polyclonal antibodies as well as recombinant proteins comprising the binding domains, as wells as fragments, including Fab, Fab′, F(ab)₂, and F(ab′)₂ fragments.

The Rsk specific inhibitory compounds/compositions of the present invention can be further combined with pharmaceutically acceptable carriers and other therapeutic compounds (such as anti-tumor agents) to provide therapeutic pharmaceutical compositions for treating a wide range of diseases that are associated with inappropriate Rsk activity. In accordance with one embodiment a composition comprising a Rsk specific inhibitor and an anti-tumor agent is provided. The composition may further include a pharmaceutically acceptable carrier. In one embodiment the Rsk specific inhibitor is an anti-sense oligonucleotide or an interfering oligonucleotide wherein the anti-sense oligonucleotide and interfering oligonucleotide comprise a nucleic acid sequence that is complementary to a nucleic acid sequence present in a Rsk gene, including Rsk1, Rsk2, Rsk3 or Rsk4.

In one aspect of the invention the presently disclosed Rsk inhibitors are used to treat various neoplastic diseases, including cancers such as prostate and breast cancer. Advantageously, since the inhibitors of the present invention inhibit Rsk specifically in situ without toxic effects, the inhibitors can also be used as therapeutic interventions in non-terminal diseased states such as epilepsy in which the MAPK signaling pathway is improperly regulated. In accordance with one embodiment, the Rsk-specific inhibitors of the present invention are used to treat cancer and neurological disorders such as epilepsy.

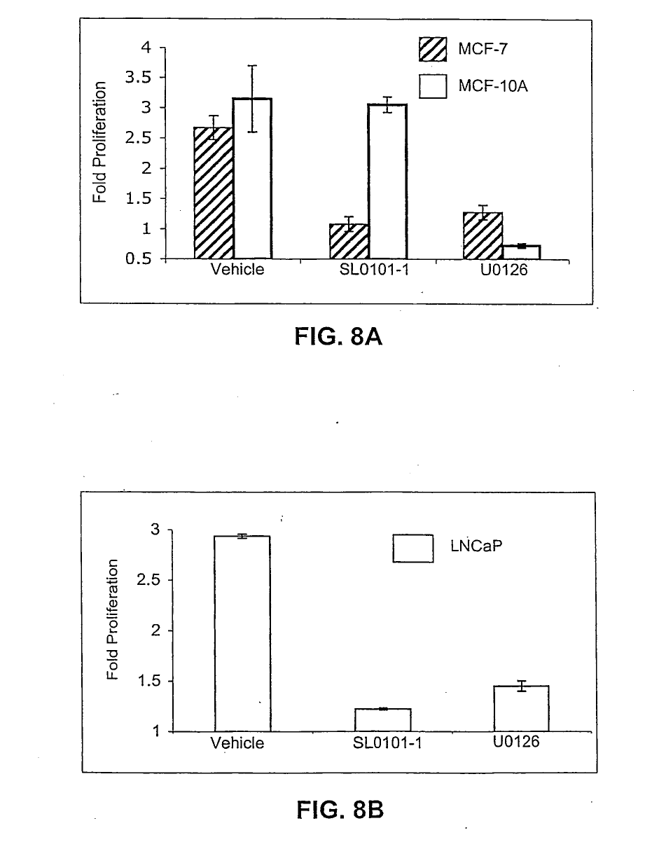

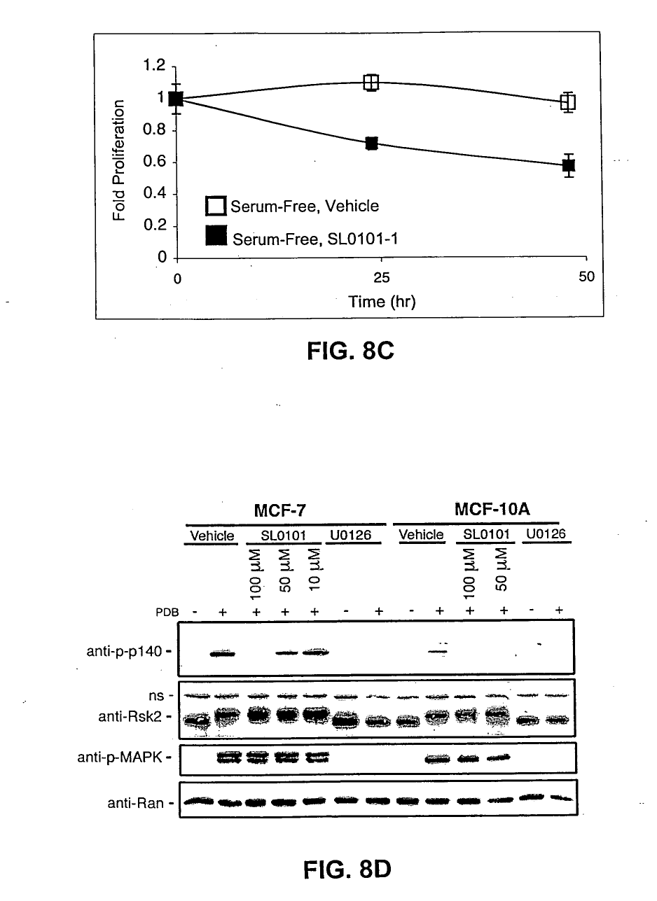

The involvement of Rsk in breast cancer has not previously been examined. However as described in detail in Example 4 the Rsk specific inhibitors of the present invention can inhibit the growth rate of MCF-7 cells, which are more representative of human cancers than the Ha-Ras transformed cell line. Remarkably, SL0101-1 inhibited proliferation of MCF-7 cells but had no effect on the growth of the normal breast cell line, MCF-10A, even though SL0101-1 prevented the PDB-induced p140 phosphorylation in MCF-10A cells (FIG. 8A). Furthermore, SL0101-1 inhibits the growth rate of MCF-7 cells at an efficacy that parallels its ability to suppress Rsk activity in vivo.

Specific inhibition of Rsk in situ was determined by incubation of MCF-7 cells in the presence or absence of increasing concentrations of extract fraction enriched in SL0101 prior to stimulation of the MAPK pathway with phorbol dibutyrate (PDB). The presence of the Rsk inhibitor eliminated phosphorylation of the Rsk substrates Estrogen Receptor alpha (ERa) and pp140 as determined using phospho-specific antibodies developed using the Rsk phosphorylation site in the ERα as the antigen. However, the inhibitor did not alter phosphorylation of Rsk by MAPK as indicated by the generation of Rsk with reduced mobility observed following SDS-PAGE. The inhibitor did not influence activation of MAPK by the MAPK Kinase, MEK as determined by the phospho-specific antibody recognizing active MAPK. Therefore, SL0101 did not inhibit the catalytic activity of Protein Kinase C (PKC), RAF, MEK, or MAPK because these kinases are essential to cause phosphorylation of Rsk in cells stimulated with PDB. Thus, SL0101 is a Rsk-specific inhibitor in situ as well as in vitro and can be used as an investigative tool for defining the function of Rsk in situ. SL0101-1 has also been found to completely inhibit the proliferation of LNCaP cells (an androgen-dependent human prostate line), see FIG. 8B. This result suggests that the Rsk inhibitors of the present invention can be used to treat prostate cancer.

It has been suggested in the literature that one of the numerous events involved in tumor initiation and progression is an increased reliance on the signaling pathway for which regulation has been compromised. This increased reliance coincides with the dormancy of alternative signaling pathways. Thus it is possible that the growth of MCF-7 cells have become dependent on the Rsk pathway rendering these cells susceptible to inhibition by SL0101-1. The growth of MCF-10A cells would not be inhibited by SL0101-1 because alternative signaling pathways regulating proliferation are intact providing numerous mechanisms for circumventing inhibition of a single signaling event. Interestingly, MCF-7 cells overexpressed Rsk2 in comparison to MCF-10A. Furthermore approximately 50% of breast cancers have elevated levels of either Rsk1 or Rsk2 compared to normal tissue. It is anticipated that the growth of tumors, such as breast cancer and prostate cancer, that overexpress Rsk, will be susceptible to inhibition by Rsk-specific inhibitors, such as SL0101-1.

Therefore one aspect of the present involves screening individuals for elevated Rsk protein levels and/or Rsk activity (relative to general population levels) as a means of identifying patients that may benefit from Rsk specific inhibitory therapy (i.e. using Rsk levels as a “therapeutic indicator”). For example individuals that have cancer or suffer from a neurological disorder may have elevated Rsk levels or activity, and it is anticipated that such patients would benefit from therapy that includes the administration of a Rsk inhibitor. In one embodiment the present Rsk specific inhibitors can be used to treat cancer (such as breast cancer) either by using the inhibitors as the sole therapeutic agent or in combination with other anti-tumor agents. Furthermore, Rsk protein levels and/or Rsk activity can be used as a therapeutic indicator, and to monitor the effectiveness of a therapeutic treatment, during or after completion of the treatment, allowing for modification of the dosage or other factors to maximize efficacy of the treatment.

Due to the association of elevated Rsk levels with cancer cells, one aspect of the present invention is directed to a method of screening individuals for neoplastic disease, such as breast and prostate cancer, by detecting the expression levels or activity of Rsk in the tissues of such patients. In one embodiment a diagnostic method for detecting neoplastic cells comprises the steps of measuring a Rsk quantification factor, in a biological sample isolated from an individual, and determining if the Rsk quantification factor is elevated relative to an internal or external standard. The Rsk quantification factor is any component that relates to the expression or activity of Rsk. In other words this may include mRNA levels or protein levels as well as enzymatic activity which may or may not correlate with Rsk protein levels. Detection of an elevated Rsk quantification factor (i.e. elevated Rsk nucleic acid quantity, Rsk protein quantity or Rsk activity) in the biological sample indicates the presence of neoplastic cells.

Typically the biological sample used for measuring the Rsk quantification factor will comprise a tissue or cell sample recovered from an individual, for example during a biopsy. However, blood or serum samples can also be screened for the presence of Rsk nucleic acid sequences or peptides. Analysis of the biological sample to quantitate the Rsk content of the sample can be conducted using standard techniques known to those skilled in the art. Overexpression of Rsk can be detected either by determining cellular nucleic acid concentrations or by determining cellular Rsk protein concentrations. This includes the use of in situ analysis as well as the purification of the Rsk nucleic acid or protein. For example, the amount of Rsk nucleic acid present in the sample can be determined using labeled complementary Rsk nucleic acid sequences and standard Southern or Northern blotting techniques or in situ hybridization techniques. Similarly, the amount of Rsk protein present in the sample can be determined through the use of antibodies that are specific for Rsk epitopes or through the use of other analytical techniques. Alternatively, in one embodiment the Rsk protein is purified from the biological sample, and the Rsk kinase activity of the recovered material is determined through the use of an in vitro kinase assay. In one embodiment the kinase activity is measured through the use of phospohospecific and/or nonphospohospecific antibodies directed against a Rsk substrate after conducting a kinase assay.

In one embodiment the Rsk activity is determined by isolating Rsk protein from the biological samples, conducting in vitro kinase assays and determining the rate of formation of phophorylated substrate. Alternatively, in one embodiment the amount of Rsk protein is determined by contacting the Rsk protein with a labeled antibody specific for Rsk protein, removing the non-bound and non-specific antibody; and quantifying the amount of label remaining to determine the amount of Rsk protein present. In another embodiment the amount of Rsk nucleic acids present in the biological sample is determined by contacting the nucleic acids of the sample with a labeled Rsk complementary nucleic acid probe, removing the non-bound and non-specific probe; and quantifying the amount of label remaining to determine the amount of Rsk nucleic acid.

The amount of Rsk detected in the biological sample is measured against an internal or external standard. For example, when an internal standard is used to determine if the Rsk nucleic acid or protein is being overexpressed, the levels detected in the recovered biological sample can be compared to the nucleic acid or protein levels of a non-Rsk gene that is expressed in the same tissue used for measuring the Rsk levels. In one embodiment a house keeping gene is selected as the internal reference, including for example, actin, Ran or some other gene whose level typically does not fluctuate in the cells selected for the biological sample (i.e. the biopsy tissue). Similarly when measuring the activity of Rsk, the detected Rsk activity in the tissue can be compared to another enzymatic activity present in the same tissue used for measuring the Rsk activity. In an alternative method of measuring Rsk levels/activity relative to an internal standard, a biological sample can be taken from both healthy tissue and the target tissue (e.g. tumor tissue) of the individual, and the Rsk levels/activity can be compared between the healthy tissue and target tissue taken from the same individual.

Alternatively, the Rsk levels/activity measured in the biological sample recovered from the patient to be screened can be compared to an external standard (i.e. a biological sample derived from another source). In one embodiment the external standard constitutes an average of Rsk levels/activity measured from one or more healthy individuals and used to establish a baseline of Rsk activity. Standard curves will be ustilized based on the tissue type and amounts of starting material used. Such standard curves will be used to determine if an individual's Rsk levels are higher than the population's average levels.

In one embodiment a diagnostic kit for detecting the presence of neoplastic cells is provided. The kit comprises reagents for detecting and quantitating the amount of Rsk or Rsk activity present in a biological sample. In accordance with one embodiment the kit comprises a Rsk quantifying agent selected from the group consisting of a Rsk specific antibody, a nucleic acid sequence complementary to a Rsk gene sequence or a Rsk substrate and reagents for conducting in vitro kinase assays. In one embodiment the antibodies or nucleic acids provided with the kit are labeled or reagents are provided for labeling the Rsk specific antibodies or nucleic acid sequences. To this end, the antibodies, nucleic acids and other reagents can be packaged in a variety of containers, e.g., vials, tubes, bottles, and the like. Other reagents can be included in separate containers and provided with the kit; e.g., positive control samples, negative control samples, buffers, etc. In one embodiment the kit is further provided with an anti-tumor agent. The kit would also be provided with instructional materials for using the reagents to quantitate a Rsk quantification factor.

As reported herein, SL0101-1 inhibited proliferation of MCF-7 cells but did not cause cell death (FIG. 8A). However, SL0101-1 when used in combination with activation of the stress pathways, e.g. by serum deprivation, significantly reduced cell viability compared to vehicle control (FIG. 8C). Thus, Rsk inhibitors may be most effective when combined with other anti-tumor agents and therapies. Rsk inhibitors may also be effective at inhibiting the growth of cancers in which the MAPK pathway is overactive as indicated by the result that SL0101-1 inhibited the proliferation of Ha-Ras transformed cells but not the parental cells. In accordance with one embodiment a method of treating a neoplastic disease involves a first step of determining if the disease is characterized by elevated Rsk activity. If such elevated Rsk activity is detected, then the patient is treated with one or more of the Rsk inhibitors of the present invention.

In accordance with one embodiment of the present invention a method is provided for treating a warm blooded vertebrate patient, including humans, afflicted by a neoplastic disease. The method comprises the steps of administering to such a patient and effective amount of a Rsk specific inhibitor. The Rsk specific inhibitor can be administered to a patient in need thereof by either administering an extract of Forsteronia refracta, an antibody specific for Rsk, an RNAi oligomer specific for Rsk, an antisense oligomer specific for Rsk or by administering a composition comprising a Rsk specific inhibitor compound having the general structure:

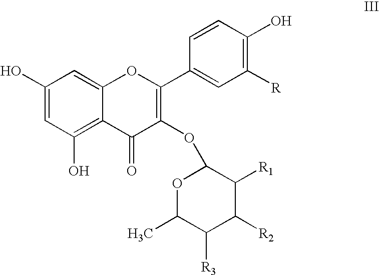

wherein R is H or OH, and R₁, R₂ and R₃ are independently selected from the group consisting of hydroxy, —OCOR₄, —COR₄, C₁-C₄ alkoxy, —O-glucoside and —O-rhamnoside and R₄ is H or C₁-C₄ alkyl. In one embodiment the compound has the structure of compound III, wherein R is H or OH, R₁ and R₂ are independently hydroxy or —OCOCH₃ and R₃ is —OCOCH₃. In another embodiment the compound has the structure of compound II or III, wherein R is H, R₁ and R₂ are independently hydroxy or —OCOCH₃ and R₃ is —OCOCH₃. These Rsk specific inhibitory compositions can be combined, or used in conjunction with, other known anti-tumor agents or therapies, such as chemotherapeutics or radiation treatments, to effectively treat cancer patients.

In accordance with one embodiment of the present invention a method for inhibiting Rsk kinase activity in a subject is provided, as a means of treating an illness associated with inappropriate Rsk activity. The inappropriate Rsk activity may constitute overexpression of Rsk protein, excessive Rsk kinase activity or it may represent the expression of Rsk activity in tissues that normally do not express Rsk activity. In accordance with one embodiment the Rsk specific inhibitors are used to treat a patient diagnosed with a disease characterized by Rsk hyperactivity, including for example, treating neoplastic disease. Alternatively, the Rsk specific inhibitors of the present invention are anticipated to have activity as antiviral agents as well as having use for treating neurological diseased states such as epilepsy. In accordance with one embodiment, a method for treating a disease characterized by elevated Rsk activity comprises the steps of administering to a human or other mammal in need thereof, a therapeutically-effective amount of a composition comprising an extract/concentrate from the tissues of Forsteronia refracta. Typically the extract or concentrate is selected from the group consisting of a food grade solvent extract, an aqueous extract (such as a methanol extract) and a dried preparation of the plant. The extract or concentrate from the tissues of Forsteronia refracta, is administered using standard routes in an amount which is effective for specifically inhibiting Rsk activity in the cells of said human or mammal.

In accordance with one embodiment a method of inhibiting Rsk activity as a means of treating a disease state comprises the steps of administering an antibody specific for Rsk, an RNAi oligomer specific for Rsk, an antisense oligomer specific for Rsk or by administering a composition comprising a compound of the general structure:

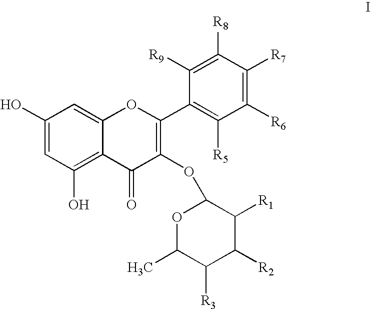

wherein R₁, R₂, and R₃, are independently selected from the group consisting of hydroxy —OCOR₄, —COR₄, C₁-C₄ alkoxy, —O-glucoside and —O-rhamnoside, R₅, R₆, R₇, R₈ and R₉ are independently selected from the group consisting of H, hydroxy —OCOR₄, —COR₄, C₁-C₄ alkoxy, —O-glucoside and —O-rhamnoside, and R₄ is H or C₁-C₄ allyl. In one embodiment, the method comprises the steps of administering a compound of Formula I, wherein R₁, R₂ and R₃ are independently selected from the group consisting of hydroxy and —OCOR₄, R₅ and R₉ are each H, R₆, R₇, and R₈ are independently selected from the group consisting of H, —OR₄, —OCOR₄, and —COR₄ and R₄ is H or methyl.

In one embodiment a method of inhibiting Rsk activity as a means of treating a disease state comprises the steps of administering a compound represented by the formula

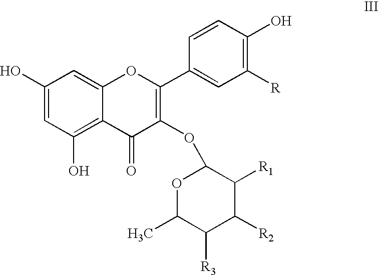

wherein R is H or OH, and R₁, R₂ and R₃ are independently selected from the group consisting of hydroxy, —OCOR₄, —COR₄, C₁-C₄ alkoxy, —O-glucoside and —O-rhamnoside and R₄ is H or C₁-C₄ alkyl. In one embodiment, the method comprises administering a compound represented by Formula II or III, wherein R is H or OH, R₁ and R₂ are independently hydroxy or —OCOCH₃ and R₃ is —OCOCH₃. In another embodiment the compound has the structure of compound II or III, wherein R is H, R₁ and R₂ are independently hydroxy or —OCOCH₃ and R₃ is —OCOCH₃.

The Rsk inhibitory compositions of the present invention can be administered either orally, parenterally, by inhalation or transdermally. In one embodiment Rsk inhibiting composition is administered locally by injection or by an implantable time release device. When administered orally, the compounds can be administered as a liquid solution, powder, tablet, capsule or lozenge. The compounds can be used in combination with one or more conventional pharmaceutical additives or excipients used in the preparation of tablets, capsules, lozenges and other orally administrable forms. When administered parenterally, for example by intravenous injection, the derivatives of the present invention can be admixed with saline solutions and/or conventional IV solutions.

One embodiment of the present invention is directed to pharmaceutical compositions comprising an Rsk inhibitory extract of Forsteronia refracta and a pharmaceutically acceptable carrier. The pharmaceutically acceptable carrier can be selected from among the group consisting of excipients, disintegrating agents, binders and lubricating agents. In a further aspect, the present invention provides a pharmaceutical composition comprising a flavonoid of the general formula I, II or III as defined above and a pharmaceutically acceptable carrier or diluent. The amount of the pharmaceutical agent suitable for administration will be in accordance with standard clinical practice.

In accordance with one embodiment of the present invention a method for screening for Rsk inhibitors is provided. The method comprises the steps of contacting a kinase substrate with Rsk in the presence of a potential inhibitory compound for a predetermined length of time under conditions normally permissive for kinase activity. The reaction is then stopped and the amount of phosphorylated substrate is quantitated. In one embodiment, a control reaction (substrate and enzyme without the potential inhibitory compound) is run simultaneously with the experimental and the amount of phosphorylation occurring in the control relative to the experimental is determined to quantify the amount of inhibition caused by the potential inhibitory compound. In a further embodiment the potential inhibitory compound is also added to a reaction containing a kinase substrate and a kinase other than Rsk to determine if the inhibitory activity of the compound is specific for Rsk.

### EXAMPLE 1

Screening Protocol

A high throughput screening (HTS) Enzyme-Linked Immunosorbent Assays (ELISAs) that can be used to screen for inhibitors of the various classes of kinases has been developed. These ELISAs can be used to obtain a robust signal-to-noise level for each of the various classes of kinases to be analyzed: Rsk represents the class of Ser/Thr kinases, focal adhesion kinase (FAK) represents the Tyr kinases, and extracellular-signal regulated kinase 2 (ERK2) represents the Pro-directed Ser kinases. The ELISAs utilize horseradish peroxidase (HRP)-conjugated phosphospecific antibodies or phosphospecific antibodies in combination with HRP-conjugated secondary antibodies. Purified, recombinant substrate (approximately 1 ug) was adsorbed to the bottom of each well in a 96 well plate. The reaction was initiated by the addition of purified, recombinant kinase (approximately 5 nM) in the appropriate buffer contain in 10 μM ATP. After 10 to 45 minutes the reaction was terminated by addition of EDTA. The wells were incubated with the appropriate antibodies, washed and the amount of chemiluminesence determined. All assays measured the initial velocity of reaction. The data was obtained from fully automated assays using the Tecan GENESIS Workstation 150 with integrated Tecan Ultra Reader.

The Z′ factor of an assay is a statistical characteristic of the quality of the assay with respect to the dynamic range and data variation of the signal measurements. A Z′ factor equal to 1 represents the ideal assay with no background and no deviation of signal, whereas a Z′. 0.5 indicates that the signal window is small to non-existent. The Z′ of the HTS ELISAs reported herein is ˜0.8, substantially higher than other HTS assays developed for the identification of kinase inhibitors. These HTS ELISAs were successfully used to screen a botanical extract library. Measurements of kinase activity with and without the presence of various extracts were compared to identify specific inhibitors of Rsk activity. Each plate in the screen contained 80 extracts as well as controls. These controls ensure that there was no plate-to-plate variation in the screen.

### EXAMPLE 2

Isolation of Rsk Inhibitors

To identify a Rsk-specific inhibitor from botanical extracts, a novel high throughput screening (HTS) Enzyme-Linked Immunosorbent Assay (ELISA) that produces luminescence as a measure of substrate phosphorylation was used (see Example 1). To discriminate extracts containing Ser/Thr kinase inhibitors from those containing nuisance compounds, a dual screen of the extracts was performed using either a constitutively active mutant of isoform 2 of Rsk (Rsk2) or the catalytic domain of the tyrosine kinase, Focal Adhesion Kinase (FAK). Of 1500 botanical extracts assayed, only one, from Forsteronia refracta, a member of the Dogbane family, inhibited Rsk2 without inhibiting FAK (FIG. 3).

Purification and structure determination of three inhibitors isolated in methanolic extracts from the plant Forsteronia refracta have been completed. The methanolic extract from wood stem and stem bark of Forsteronia refracta was applied to a polyamide 6S column, which was washed successively with H₂O, 1:1H₂O— MeOH, 9:1 CH₂Cl₂—MeOH, 1:1 CH₂Cl₂—MeOH and 9:1 MeOH—NH₄OH to afford five fractions. The 9:1 CH₂Cl₂—MeOH and 1:1 CH₂Cl₂—MeOH fractions showed stronger inhibition of Rsk than the starting material. The 1:1 CH₂Cl₂—MeOH fraction was further fractionated on a diol gel column. The column was eluted successively with CH₂Cl₂, 99:1 CH₂Cl₂—MeOH, 95:5 CH₂Cl₂—MeOH, 90:10 CH₂Cl₂—MeOH and MeOH to give five fractions. Among these, the 95:5 CH₂Cl₂—MeOH, 90:10 CH₂Cl₂—MeOH and MeOH fractions showed the same or stronger activity than the starting material. The 95:5 CH₂Cl₂—MeOH fraction was fractionated repeatedly on a C₁₈ reverse phase HPLC column (250′10 mm); elution was carried out with 65:35 MeOH—H₂O and UV detection was at 265 nm. Two compounds, SL0101-1 and SL0101-2 were obtained as amorphous pale yellow powders. On the basis of its ¹H NMR spectrum and positive APCI-MS, SL0101-1 was found to be kaempferol 3-α-L-(3″,4″-diacetyl)rhamnopyranoside and SL0101-2 was proved to be kaempferol 3-α-L-(2″,4″-diacetyl)rhamnopyranoside.

The 90:10 CH₂Cl₂—MeOH fraction from the above diol column was also fractionated repeatedly on a C₁₈ reverse phase HPLC column using 45:55H₂O—MeOH as the eluant and UV detection at 275 nm. The active constituent, SL0101-3, was obtained as an amorphous powder. On the basis of its ¹H NMR and ¹³C NMR data, the compound was found to be kaempferol-3-α-L-(4″-acetyl)rhamnopyranoside (SL0101-3).

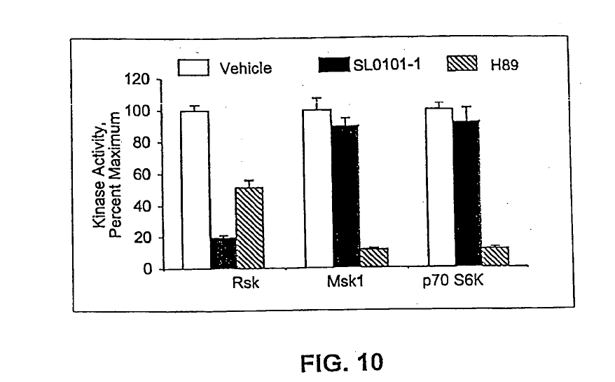

The in vitro IC50 was determined to be less than 100 nM for SL0101-1 (FIG. 2), whereas the IC50 of kaempferol, the flavonoid constituent of SL0101-1, was determined to be 15 μM for Rsk (FIG. 5). Therefore, the rhamnose moiety of SL0101-1 greatly increases the affinity for Rsk. Purified SL0101-1 is specific for inhibition of Rsk activity compared to p70 S6K and Msk1 and is competitive with respect to ATP. The specificity of purified SL0101-1 for Rsk is indicated by the data presented in FIG. 10. That data represent experiments wherein vehicle or inhibitor (5 μM) was added to the kinase mix containing 5 μM of the indicated purified kinases. The reaction proceeded for 30 mins at room temperature and the data normalized to the kinase activity obtained in the presence of vehicle. The ATP concentration was 10 μM. Phosphorylation of the substrate was detected by ELISA.

Methods

Polyamide 6S (pour density 0.25 g/mL, a product of Riedel-de Haen, Germany) was obtained from Crescent Chemical Co. Lichroprep diol (40-63 μm) is a product from EM Industries, Inc. A Kromasil C₁₈ reverse phase column (250×10 mm, 5 μm) for HPLC was obtained from Higgins Analytical Inc. ¹H NMR spectra were measured on General Electric QE 300, GN-300 NMR or Varian unity INOVA-500 spectrometers. Mass spectra were recorded on a Finnigan MAT 4600 mass spectrometer.

Wood stem and stem bark of Forsteronia refracta was soaked three times with methanol at room temperature. The resulting methanol solutions were combined and concentrated under diminished pressure to afford the crude extract. The crude extract (888 mg) was applied to a 40-g polyamide 6S column, which was washed successively with 150 mL each of H₂O, 1:1H₂O— MeOH, 9:1 CH₂Cl₂—MeOH, 1:1 CH₂Cl₂—MeOH and 9:1 MeOH—NH₄OH to afford five fractions. The 1:1 CH₂Cl₂—MeOH fraction (126.7 mg) showed stronger inhibition of Rsk than the starting original extract. The 1:1 CH₂Cl₂—MeOH fraction was further fractionated on a 30-g diol gel column. The column was washed, respectively, with 150 mL each of CH₂Cl₂, 99:1 CH₂Cl₂—MeOH, 95:5 CH₂Cl₂—MeOH, 90:10 CH₂Cl₂—MeOH and MeOH to give five fractions. Among these, the 95:5 CH₂Cl₂—MeOH (38.1 mg) fraction showed the same or stronger activity than the starting material. The 95:5 CH₂Cl₂—MeOH fraction (4 mg) was fractionated repeatedly on a C₁₈ reverse phase HPLC column (250×10 mm); with elution was 65:35 MeOH—H₂O at a flow rate of 3 mL/min, with UV detection at 265 nm. SL0101-1 (2 mg) was obtained as an amorphous pale yellow powder. From the ¹H NMR and positive APCI-MS, SL 0101-1 was confirmed to be kaempferol 3-α-L-(3″,4″-diacetyl)rhamnopyranoside.

### EXAMPLE 3

Analysis of Inhibitor Site of Action

Rsk contains two non-related kinase domains in a single polypeptide chain. The amino-terminal kinase domain (NTKD) is mostly closely related to p70 S6K whereas the carboxyl-terminal kinase domain (CTKD) is most similar to the calmodulin-dependent protein kinases. Regulation of Rsk is complex and requires a cascade of phosphorylations resulting from the actions of MAPK, the CTKD of Rsk itself, and 3-phosphoinositide-dependent protein kinase-1. The NTKD phosphorylates exogenous substrates whereas the only known function of the CTKD is autophosphorylation.

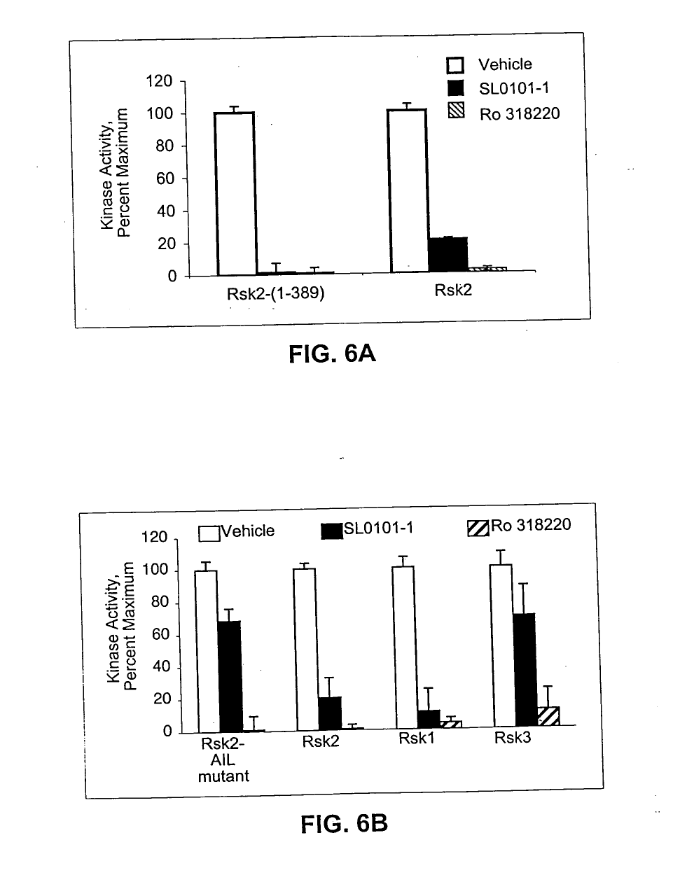

To determine the domain inhibited by SL0101-1, the ability of SL0101-1 to inhibit full-length or a truncation mutant of Rsk2 containing only the NTKD (Rsk2(1-389)) was compared (FIG. 6A). HA-tagged Rsk2 and an HA-tagged truncation mutant containing the NTKD (Rsk2 with only the first 389 amino acids) were transfected into baby hamster kidney 21 (BHK21) cells. The HA-tagged proteins were immunoprecipitated from lysates of EGF-stimulated cells. Kinase assays were performed using immobilized substrate. The extent of phosphorylation was determined using phosphospecific antibodies directly labeled with horseradish peroxidase (HRP)-conjugated or phosphospecific antibodies in combination with HRP-conjugated secondary antibodies. All assays measured the initial reaction velocity and maximum activity was measured in the presence of vehicle. Assays were performed in the presence of vehicle, 2 μM SL0101-1 or a known non-specific inhibitor, 2 μM Ro 318220. SL0101-1 potently inhibited the isolated Rsk2 NTKD, indicating that inhibition of Rsk occurs through competition with ATP for the nucleotide-binding site of the NTKD.

Alignment of residues forming the ATP-binding pocket of Rsk with those of p70 S6K, Msk1 and PKA revealed a difference in contacts to the adenosine ring. Indeed, the sequence 145LILDFLRGGDLFT157 (SEQ ID NO: 1), referred to as the adenosine-interacting loop (AIL), is unique to the Rsk family. To examine the importance of this region in determining SL0101-1 specificity, a mutant Rsk2 was created in which the p70 S6K AIL (147LILEYLSGGELFM159; SEQ ID NO: 2) replaced that of Rsk2 (Rsk2-AIL mutant). SL0101-1 was ˜3-fold less effective in inhibiting the Rsk2-AIL mutant in comparison to wild type Rsk2 (FIG. 6B). Therefore, the unique adenosine-interacting loop of Rsk is a major determinant for SL0101-1 binding. However, the mutation did not completely abolish inhibition by SL0101-1, indicating the presence of additional points of contact.

To determine whether the unique adenosine-interacting loop is sufficient for SL0101-1 specificity, the isozyme specificity of SL0101-1 was examined. The primary structure of the NTKDs of the Rsk isoforms 1-3 are highly related, sharing 87% identity and each contain the unique adenosine-interacting loop. HA-tagged proteins were immunoprecipitated from the lysates of EGF-stimulated BHK21 cells transiently transfected with the indicated HA-tagged constructs (Rsk1, Rsk2 and Rsk3). Assays were performed as described immediately above. Remarkably, although SL0101-1 potently reduced Rsk1 and Rsk2 activity, the Rsk3 activity was only partially inhibited (FIG. 6B). Thus, the adenosine interacting loop is necessary but not sufficient for conferring SL0101-1 specificity and the Rsk1 and Rsk2 isoforms must have additional contact points that increase the affinity for SL0101-1. These results further attest to the high specificity of SL0101-1.

### EXAMPLE 4

Inhibition of Cell Proliferation by Rsk Inhibitors

To determine whether SL0101-1 inhibits Rsk in intact cells, phosphorylation of p140, a Rsk substrate of unknown function, was examined in a human breast cancer cell line, MCF-7. MCF-7 and MCF-10A cells were pre-incubated with vehicle, 50 μM U0126 or the indicated concentration of SL0101-1 for 3 hr (FIG. 8D). Cells were treated with 500 nM PDB for 30 min prior to lysis. Protein concentration of lysates was measured and lysates were electrophoresed, transferred and immunoblotted. Equal loading of lysate is demonstrated by the anti-Ran immunoblot. Pre-incubation of cells with 100 μM SL0101-1 abrogates phorbol dibutyrate (PDB)-induced p 140 phosphorylation as does 50 μM U0126, a MEK inhibitor. SL0101-1 does not effect the phosphorylation of Rsk2, as indicated by the reduced electrophoretic mobility of Rsk2, nor the activation of MAPK, as detected by the anti-active MAPK antibody (see FIG. 8D). Therefore, SL0101-1 does not inhibit upstream kinases necessary for PDB-stimulated Rsk phosphorylation, namely MAPK, MEK, Raf and PKC. These data indicate that SL0101-1 is an effective and specific Rsk inhibitor in intact cells.

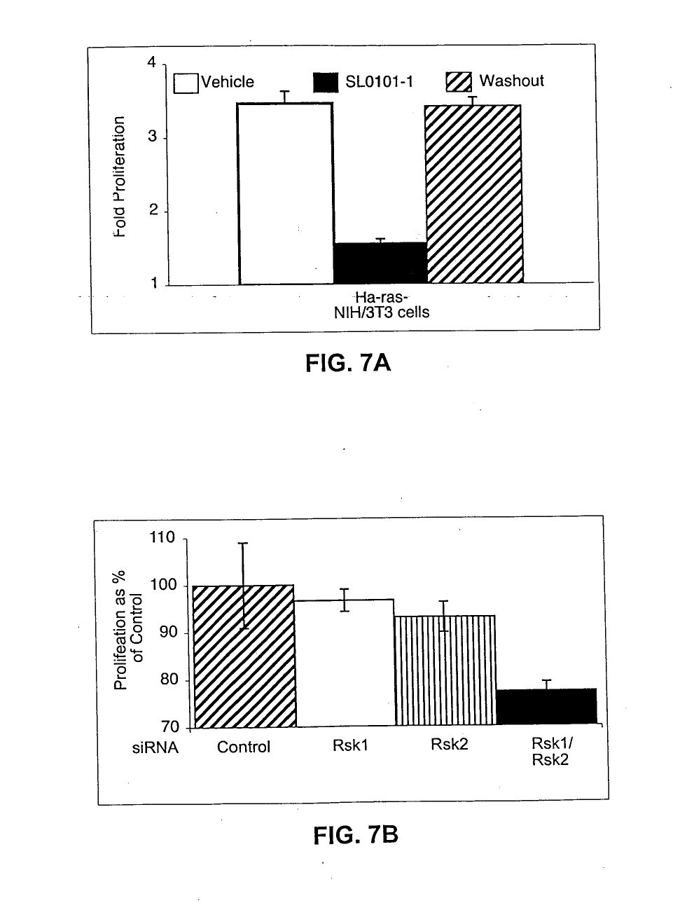

The importance of MAPK to proliferation and oncogenesis is well established. However, the role that Rsk plays in these processes has not been examined. Therefore, the effect of SL0101-1 on proliferation of Ha-Ras transformed NIH/3T3 cells and the parental cell line was determined. SL0101-1 decreased the growth rate of the transformed cells but had little effect on proliferation of the parental line (FIG. 4). SL0101-1 produced striking morphology changes in the transformed cells but not in the parental cell line. The vehicle control treated Ha-Ras transformed cells were elongated whereas in response to SL0101-1 the cells became much larger and flatter, appearing more like the parental cells, or like Ha-Ras transformed cells treated with U0126. Removal of SL0101-1 resulted in growth of the transformed cells (see FIG. 7A) and a reversion to their elongated phenotype. These results demonstrate that SL0101-1 can penetrate intact cells, but is not toxic and preferentially inhibits the growth of oncogene-transformed cells compared to the parental cells.

Whether or not SL0101-1 could inhibit the growth rate of MCF-7 cells, which are more representative of human cancers than the Ha-Ras transformed cell line, was also investigated. Remarkably, SL0101-1 inhibited proliferation of MCF-7 cells but had no effect on the growth of the normal breast cell line, MCF-10A (FIG. 8A), even though SL0101-1 prevented the PDB-induced p140 phosphorylation in MCF-10A cells (FIG. 8D). Furthermore, SL0101-1 inhibits the growth rate of MCF-7 cells at an efficacy that parallels its ability to suppress Rsk activity in vivo.

Reduction of Rsk1 and Rsk2 levels was also accomplished using short, interfering RNAs (siRNA). Specifically, duplex siRNAs to a sequence in the bluescript plasmid (Control) or to Rsk1 and Rsk2 were transfected into MCF-7 cells. The sense strand for Rsk1 has the sequence AAGAAGCUGGACUUCAGCCGU (SEQ ID NO: 5), whereas the sense strand for Rsk1 has the sequence AACCUAUGGGAGAGGAGGAGA (SEQ ID NO: 6). Medium was replaced 24 hr post-transfection and the cells incubated for an additional 48 hr prior to measuring cell viability. A combination of siRNAs to both Rsk1 and Rsk2 was effective in reducing MCF-7 proliferation (FIG. 7B). The siRNAs were not as effective at inhibiting growth as SL0101-1, however Rsk1 and Rsk2 expression was not completely eliminated and only about 70% of the cells were transfected. Nonetheless, these results strongly support observations that both Rsk1 and Rsk2 are important in MCF-7 proliferation.

As further support of the specificity of SL0101-1 action, U0126, the MEK inhibitor, halted proliferation of both MCF-7 and MCF-10A cells (FIG. 8A). Ro 318220 (500 nM), a potent but non-specific PKC inhibitor, which inhibits Rsk as well as a number of other AGC kinase family members also attenuated proliferation of both MCF-7 and MCF-10A cells. Moreover, kaempferol, the flavonoid constituent of SL0101-1 slows growth of MCF-10A and MCF-7 cells to the same extent. Therefore, unlike the action of these other kinase inhibitors, SL0101-1 selectively halts proliferation of cancer cells without affecting normal cells.

Methods.

Kinase Assays. Glutathione-5-transferase (GST)-fusion protein (1 g) containing the sequence—RRRLASTNDKG (SEQ ID NO: 3, for serine/threonine kinase assays) or -VSVSETDDYAEIIDEEDTFT (SEQ ID NO: 4, for tyrosine kinase assays) was adsorbed in the wells of LumiNunc 96-well polystyrene plates (MaxiSorp surface treatment). The wells were blocked with sterile 3% tryptone in phosphate buffered saline and stored at 4° C. for up to 6 months. Kinase (5 nM) in 70 μl of kinase buffer (5 mM 25-glycerophosphate pH 7.4, 25 mM HEPES pH 7.4, 1.5 mM DTT, 30 mM MgCl₂, 0.15 M NaCl) was dispensed into each well. Compound at indicated concentrations or vehicle was added, and reactions were initiated by the addition of 30 μl of ATP for a final ATP concentration of 10 μM unless indicated otherwise. Reactions were terminated after 10 to 45 min by addition of 75 μl of 500 mM EDTA, pH 7.5. All assays measured the initial velocity of reaction. After extensive washing of wells, polyclonal phosphospecific antibody developed against the phosphopeptide and HRP-conjugated anti-rabbit antibody (211-035-109, Jackson ImmunoResearch Laboratories) were used to detect serine phosphorylation of the substrate. HRP-conjugated anti-phospho-tyrosine antibody (RC20, BD Transduction Laboratories) was used for phospho-tyrosine detection. His-tagged active Rsk and FAK were expressed in Sf9 cells and purified using NiNTA resin (Qiagen). Baculovirus was prepared using the Bac-to-Bac® baculovirus expression system (Invitrogen). PKA was bacterially expressed and activated as described (Anal. Biochem. 245, 115-122 (1997)). Active Mskl and p70 S6 kinase was purchased from Upstate Biotechnology. Immunoprecipitation and kinase assays were performed as previously described (Poteet-Smith et al., J. Biol. Chem, 274, 22135-22138 (1999) using the immobilized GST-fusion proteins and ELISAs as above.

Cell Culture. For proliferation studies cells were seeded at 2500 to 5000 cells per well in 96 well tissue culture plates in the appropriate medium as described by American Type Culture Collection. After 24 hr, the medium was replaced with medium containing compound or vehicle as indicated. Cell viability was measured at indicated time points using CellTiter-Glom assay reagent (Promega) according to manufacturer's protocol. For in vivo inhibition studies, cells were seeded at 2.5×10⁵ cells/well in 12 well cell culture clusters. After 24 hr, the cells were serum starved for 24 hr then incubated with compound or vehicle for 3 hr prior to a 30 min PDB stimulation. Cells were lysed as previously described (J. Biol. Chem. 273, 13317-13323 (1998)). The lysates were normalized for total protein, electrophoresed and immunoblotted. For cell imaging, Ha-Ras-transformed NIH/3T3 cells were seeded on LABTEK II chamber slides (Nalge) at a density of 1×10⁴ cells/well. After 24 hr, fresh medium was added the indicated compounds or vehicle. Images were taken 48 hr after treatment at a magnification of 20×.

Gene Silencing. Custom oligonucleotides to Rsk1 (AAGAAGCUGGACUUCAGCCGU; SEQ ID NO: 5 and Rsk2 (AACCUAUGGGAGAGGAGGAGA; SEQ ID NO: 6) mRNA (Dharmacon Research Inc.) and TransIT-TKO® siRNA Tranfection Reagent (MIR2150, Mirus Corporation) were used for the gene silencing studies. MCF-7 cells were seeded at a density of 1.25×10⁴ cells per well in 24 well cell culture clusters. After 24 hr, fresh medium was added containing 25 nM oligonucleotide and transfection reagent according to manufacturer's protocol. The transfection medium was replaced after 24 hr. Cells were incubated for an additional 48 hr prior to cell viability measurement.

Breast tissue analysis. Frozen tissue samples were ground using mortar and pestle under liquid nitrogen. Ground tissue was added to heated 2-×SDS loading buffer and boiled for 3 min. Protein concentration of lysates was measured and lysates were electrophoresed on SDS-PAGE and immunoblotted.

### EXAMPLE 5

Rsk Inhibitors Inhibit Proliferation of Prostate Cancer Cell Line

Prostate cancer is the second most common cancer in men and approximately one in six men will be diagnosed with the disease. Early stage prostate cancer is frequently dependent on the hormone, androgen. Androgen action is mediated through interaction with the androgen receptor, a member of the superfamily of ligand-activated transcription factors. Inhibition of androgen receptor activity by pharmacological or surgical interventions that reduce androgen concentration can result in prostate tumor regression. However, with relatively high frequency the tumors become androgen-independent, which often leads to a fatal outcome. Treatment options are confined to conventional chemotherapy because of the lack of specific drug targets associated with androgen-independent prostate cancer. Thus, elucidation of the mechanisms that result in the transition of prostate cancer from an androgen-dependent to androgen-independent state will greatly facilitate the identification of more effective therapies.

An increase in mitogen-activated protein kinase (MAPK) activity has been correlated to prostate cancer progression in human tumors. This enhanced activity is most likely due to the increase in growth factors and receptors that are known to occur. Activation of growth factor receptors enhance MAPK activity via a kinase cascade that is regulated by the small GTP-binding protein, p21Ras. The family of p90 ribosomal S6 kinases (Rsks), which are Ser/Thr protein kinases, function as downstream effectors of MAPK. The biological actions of the Rsks are not well characterized partly because until recently there were no known inhibitors of Rsk that did not also inhibit MAPK activity.

The first Rsk-specific inhibitor, SL0101-1 has now been isolated. As described in Example 4, SL0101-1 inhibits the proliferation of the breast cancer cell line, MCF-7, without preventing the proliferation of a normal breast cell line, MCF-10A. Furthermore, in NIH 3T3 fibroblasts, SL0101 reduces the growth of a Ha-Ras-transformed line but not of the untransformed parental cells. It is believed that SL0101 specifically inhibits the growth of transformed cells because transformed cells preferentially depend on the Rsk pathway to regulate proliferation. These results provide the first demonstration that the Rsk family through the regulation of its downstream effectors is involved in the control of cancer cell proliferation. Relatively few downstream effectors of Rsk have been identified. However, Rsk is known to phosphorylate and regulate the activity of a number of transcription factors, the pro-apoptotic protein, BAD, and the mitotic checkpoint kinase, BUB1. Determining which Rsk substrates play a key role in cancer cell proliferation will undoubtedly lead to the discovery of novel drug targets for cancer therapy.

The ability of SL0101-1 to inhibit the proliferation of the androgen-dependent human prostate line, LNCaP was tested. SL0101-1 completely inhibits the proliferation of LNCaP cells (FIG. 8B). This result suggests that the LNCaP line is primarily dependent on Rsk activity for growth. To investigate the Rsk signal transduction pathway in the LNCaP line, a phosphospecific antibody to a Rsk phosphorylation motif (RPM) was produced. Only a few downstream effectors of Rsk have been identified and therefore, applicants anticipated that an anti-RPM antibody would be a very effective tool for identifying novel Rsk substrates in vivo. An anti-RPM antibody has previously been reported that recognizes the Rsk substrate, p 140, a protein of unknown function. In agreement with these results SL0101-1 was observed to decrease the phosphorylation of p140 with an efficacy that paralleled its ability to inhibit LNCaP proliferation (see FIGS. 8B and 8D). All lysates were normalized to each other using an anti-Ran antibody. Ran is used for normalization based on the observations that it is a general housekeeping protein, the activity or expression levels of which are not known to vary in any disease state.

Malignant transformation and progression in human cancers are frequently associated with over-abundance or increased activity of proteins that are involved in normal cellular processes. As reported herein, Rsks have been found to be overexpressed in many human breast and prostate cancers, as compared to normal breast and prostate tissue. Lysates were made from the various samples and normalized to each other using an anti-ran antibody. Examination of Rsk1 and Rsk2 expression in 22 breast cancer samples and 4 normal samples revealed that >50% of the breast cancer tissues have higher Rsk expression than the normal samples. Rsk1 and Rsk2 expression was also examined in 4 prostate cancers, 5 normal and 5 benign hyperplastic (BPH) samples. In general, the cancer tissues have higher levels of Rsk expression than the normal and BPH tissue with the exception of one normal sample. However, this sample was removed from tissue that was adjacent to cancerous tissue. Interestingly, phosphorylation of p140 could be detected in normal prostate tissues except for the one normal tissue that also contained a higher level of Rsk1 expression. Under the electrophoretic conditions used in this experiment the phosphorylated p140 migrates as a doublet. The cancerous tissue was obtained from tumors with Gleason scores >7, which indicates that the samples are of advanced prostate cancers.

The breast and prostate lysates were also immunoblotted with anti-pan ERK antibody, which recognizes both the active and inactive forms of p42 and p44 MAPK. The relative levels of p42 and p44 MAPK varied considerably between the samples but did not correlate with the extent of Rsk overexpression. Thus Rsk overexpression is not merely a reflection of overexpression of various members of the MAPK pathway. These results indicate that both Rsk1 and Rsk2 activity is higher in human breast and prostate cancer tumors than in normal human breast tissue. These results support the use of Rsk as a good drug target for breast and prostate cancer, and other cancer types.

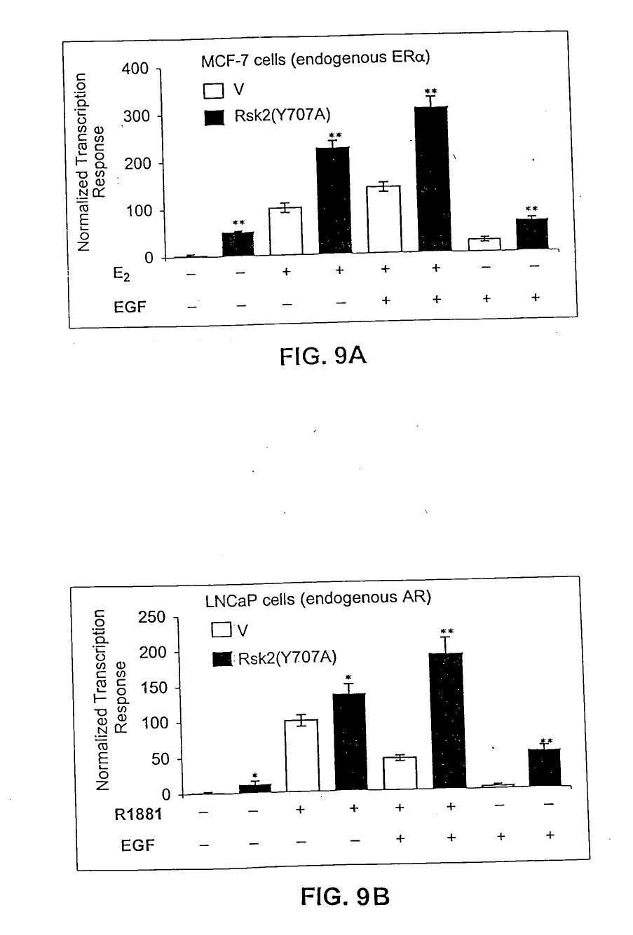

Overexpression of the isoform 2 of the Rsk family (Rsk2) also enhances the transcriptional activity of the estrogen receptor (ERα) and the androgen receptor (AR). A constitutively active mutant of Rsk2 was prepared to allow for the study of Rsk2's role in ER-mediated transcription in the absence of active MAPK. Rsk2 enhanced both ligand-dependent and ligand-independent ER-mediated transcription in MCF-7 cells, a human breast cancer cell line (FIG. 9A). Additionally, Rsk2 enhances the ligand-dependent and lingand-independent transcription of AR-mediated transcription in LNCaP cells, a human prostate cancer cell line (FIG. 9B). These results are significant because they suggest that the enhanced Rsk expression observed in breast and prostate cancer cells may increase ERα or AR transcriptional activity. Increased activities of the ERα and AR are known to be important in the etiology of some breast and prostate cancers, respectively.

### EXAMPLE 6

Proposed Synthetic Schemes for Preparing Rsk Inhibitors

Abbreviations used in Examples 6-9 are as follows: TBDPS=tert-butyldiphenylsilyl, THF=tetrahydrofuran, EDCI=1-(3-dimethylaminopropyl)-3-ethylcarbodiimide, DMAP=4-dimethylaminopyridine, TSOH=4-toluene sulfonic acid, DMF=dimethylformamide, Bn=benzyl, MTBE=methyl tert-butyl ether.

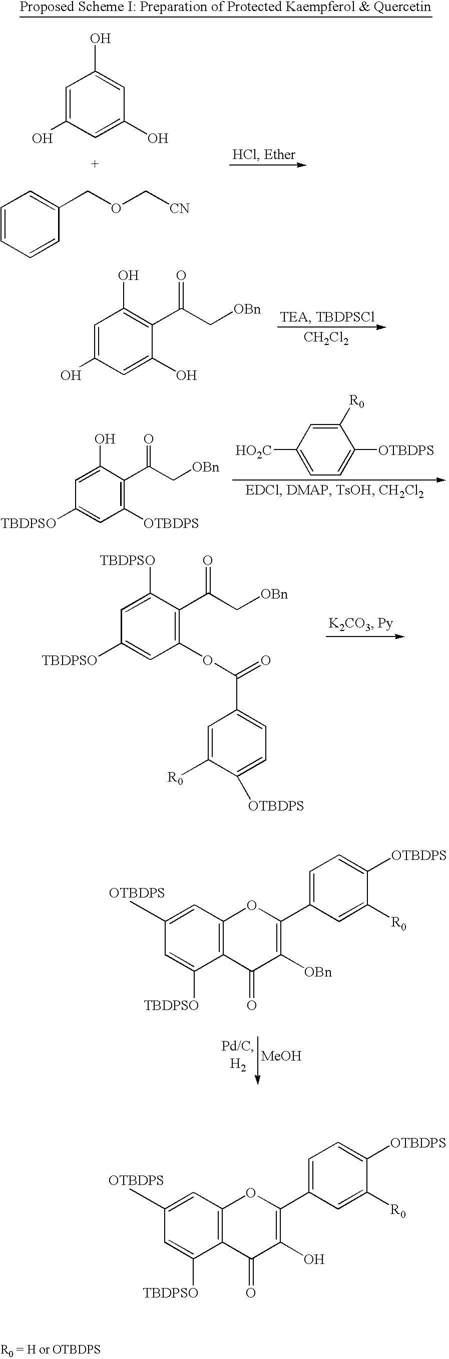

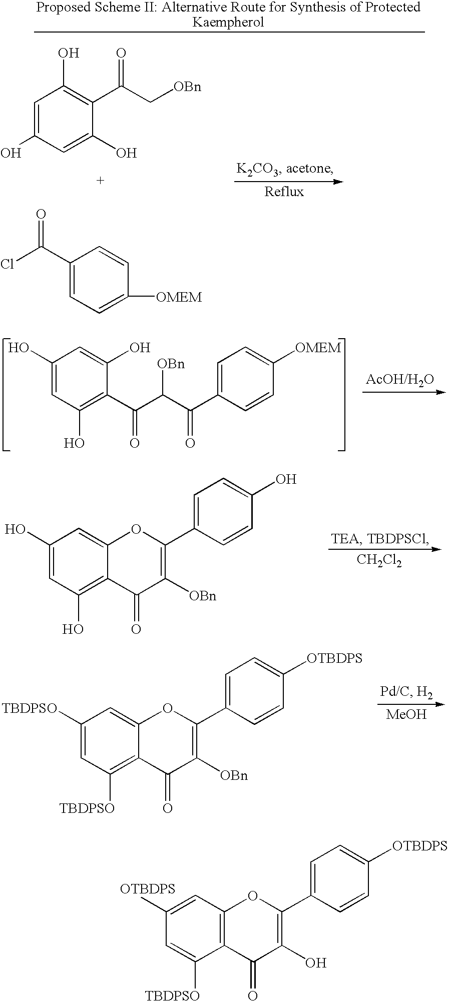

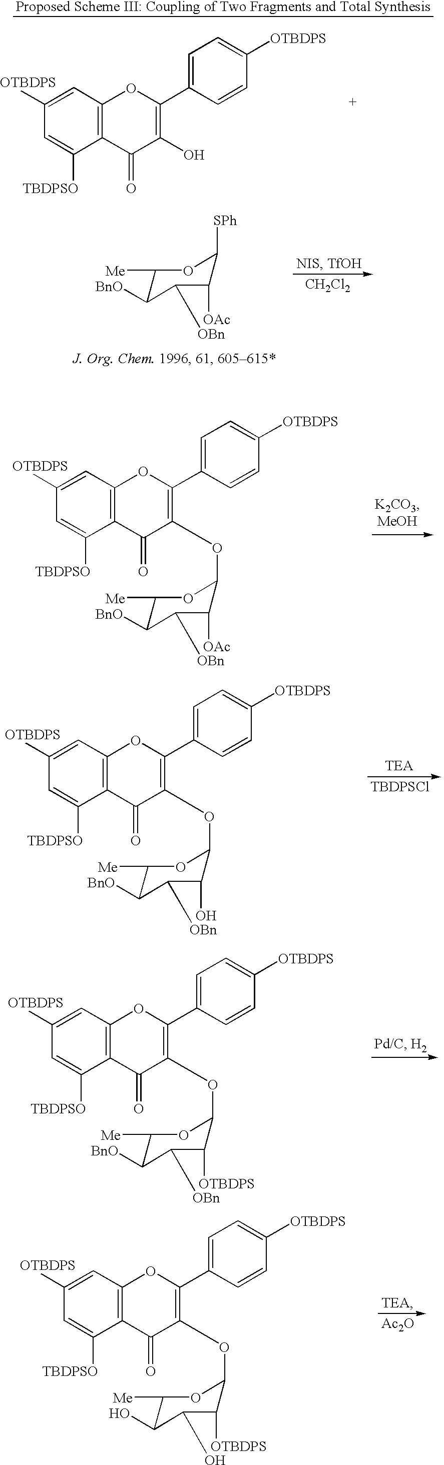

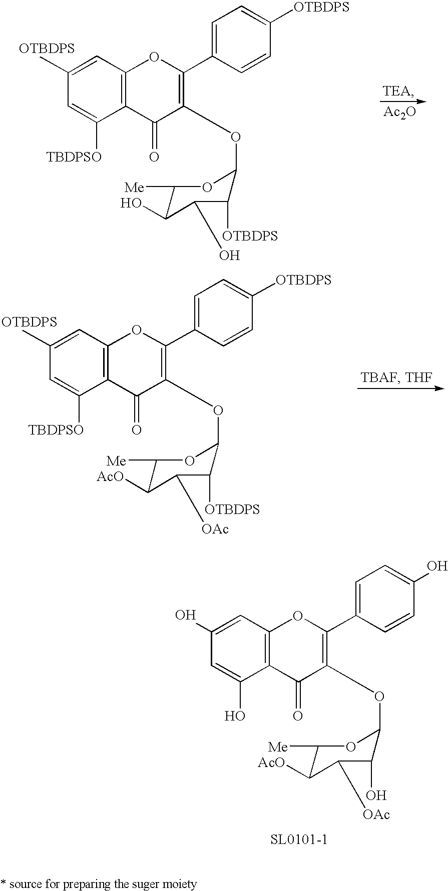

### EXAMPLE 7

Synthesis of the Protected Kaempferol (10)

The synthesis for the Kaempferol half of SL0101-1 is outlined as follows:

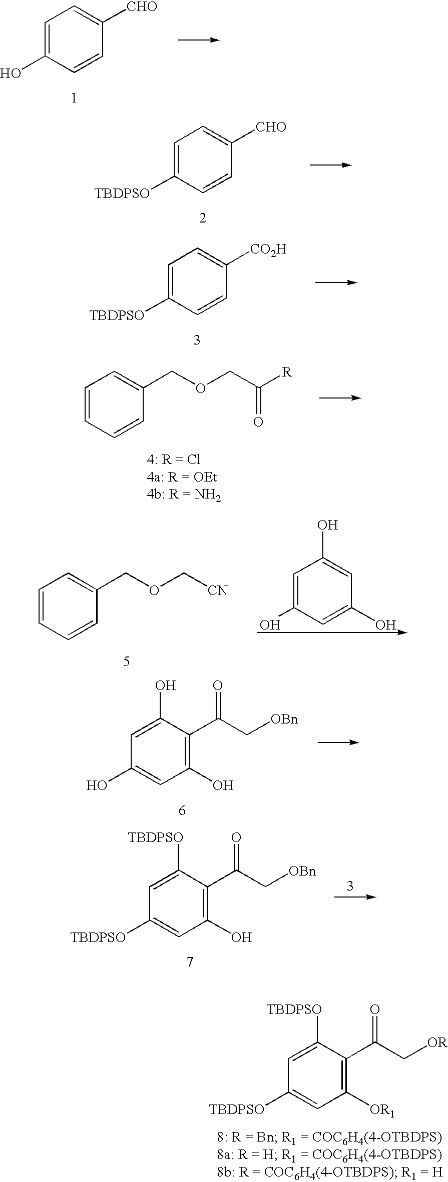

Treatment of commercially available 1 (20 g) with tert-butyldiphenylsilyl chloride (TBDPSCl) and imidazole in THF/CH₂Cl₂ gave, after chromatographic purification, 2 (47.3 g, 80%). This compound was characterized by ¹H NMR and MS. Oxidation of 2 (21.8 g and 25 g) using sodium chlorite gave 3 (50 g total, quantitative yield). The product was characterized by ¹H and ¹³C NMR, and by MS.

Benzyl alcohol (50 g) on treatment with NaH (1.2 equiv) and ethyl bromoacetate (1 equiv) in THF gave 4a (32 g, 36%), which was characterized by both ¹HNMR, and by MS. Scale up of this reaction yielded 100 g of 4a. Reaction of 4a (5 g) with NH₄OH at 0° for 5 h in CH₂Cl₂ gave amide 4b (4.3 g, 96%), which was characterized by ¹H NMR and MS. A repeat of this experiment on 45 g of 4a gave 38 g (94%) of 4b. Dehydration of 4b (4.2 g) using POCl₃ in acetonitrile gave 5 (1.75 g, 47%), which was characterized by ¹H NMR, ¹³C NMR and MS. A repeat of this experiment on 38 g of 4b gave an additional 15.75 g (47%) of 5. Coupling of 5 (5 g) and phloroglucinol in MTBE with HCl gas bubbling at 0° C. for 3 h gave 6 (2.6 g, 56%), which was characterized by ¹H NMR, ¹³C NMR and MS. Selective protection of 6 (0.5 g) using TBDPSCI (2.5 equiv) and Et₃N (2.5 equiv) in CH₂Cl₂ at room temperature for 16 h gave 7 (1.2 g, 85%), which was characterized by ¹H NMR and MS. Scale-up of this experiment on 2 g of 6 gave an additional 3.4 g (62%) of 7. Condensation of 7 (1.4 g) with 3 (1.35 equiv) in CH₂Cl₂ [EDCI (1.5 equiv), DMAP (0.35 equiv), TsOH (0.35 equiv.] at room temperature for 24 h gave 8 (1.5 g, 72%), which was characterized by ¹H NMR. Scale up gave 35 g of purified 8. Compound 8 (6 g) was debenzylated using Rb/C as a catalyst (H₂, 60 psi, EtOAc, rt, 24 h) to give 8a (1.8 g, 33%) along with 2.9 g (53%) of the trans-esterified (migration of benzoyl group R₁) product 8b. Both the intermediates 8a and 8b were characterized by ¹H NMR.

### EXAMPLE 8

Synthesis of the Protected Rhamnose (20)

The synthesis for the Rhamnose half of SL0101-1 is outlined as follows:

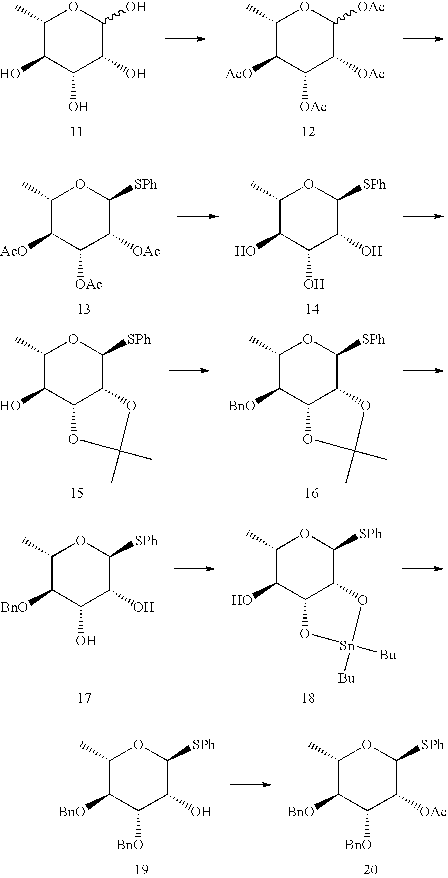

Reaction of L-rhamnose 11 (50 g) with acetic anhydride (6 equiv), triethylamine (8 equiv) and catalytic 4-dimethylaminopyridine (0.1 equiv) in CH₂Cl₂ at room temperature for 16 h gave 90 g (98%) of the tetraacetate 12, which was characterized by ¹H NMR and MS and was taken to the next step without further purification. Scale up yielded 260 g of 12. Treatment of 12 (150 g) with thiophenol (1.1 equiv) in the presence of SnCl₄ (0.7 equiv) in CH₂Cl₂ at 0° C. for 5 h gave 13 [56 g (pure), 110 g (with ˜10% impurity)], which was characterized by both ¹H NMR and MS. Deacetylation of 13 (56 g) using catalytic K₂CO₃ (0.2 equiv) in THF/MeOH (1:1) at room temperature for 16 h provided triol 14 (35 g, 93%), which was characterized by ¹H NMR and MS.

Treatment of 14 (0.3 g) using 2,2-dimethoxypropane with catalytic amount of p-toluenesulfonic acid gave 15 (0.3 g, 86%) as a single anomer, which was characterized by both ¹H NMR and MS. Scale-up of this reaction on 34 g of 14 gave an additional 38 g (97%) of 15. O-Benzylation of 15 (0.3 g) with NaH (1.74 equiv) and benzyl bromide (1.05 equiv) in DMF provided the benzyl ether 16 (0.35 g, 89%), which was characterized by ¹H NMR. A repeat of this experiment on 38 g of 15 gave 41 g (83%) of 16. Treatment of 16 (3 g) with trifluoroacetic acid in MeOH at 50° C. for 16 h gave diol 17 (2.5 g, 93%), which was characterized by ¹H NMR and MS. A repeat of this experiment on 10 g of 16 gave 8.5 g (95%) of 17. Selective O-benzylation of diol 17 (2.5 g) following a literature procedure (n-Bu₂SnO, toluene, Dean-Stark, reflux, 4 h to give 18, then n-Bu₄NBr, BnBr, 50° C., 5 h) gave 19 (2.6 g, 82%), which was characterized by both ¹H NMR and MS. Treatment of 19 (2.5 g,) with acetic anhydride and pyridine gave the acetate 20 (rhamnose part of the molecule) (2.5 g, 91%), which was characterized by ¹H NMR and MS.

Scale-up of the above reactions to get acetate 20 (˜20 g) was conducted as follows. Treatment of 16 (25 g) with trifluoroacetic acid in MeOH at 50° C. for 16 h gave diol 17 (21 g, 94%), which was characterized by ¹H NMR and MS. Selective O-benzylation of diol 17 (29.5 g) following a literature procedure (n-Bu₂SnO, toluene, Dean-Stark, reflux, 4 h to give 18, then n-Bu₄NBr, BnBr, 50° C., 5 h) gave 19 (31 g, 83%), which was characterized by both ¹H NMR and MS. Treatment of 19 (30 g) with acetic anhydride and pyridine gave the acetate 20 (29.3 g, 89%) required for the coupling reaction with 10. The product was characterized by ¹H NMR and MS.

### EXAMPLE 9

Coupling of the Kaempferol and Rhamnose Moieties

The coupling reaction between compounds 20 and 8a to generate SL0101-1 is outlined as follows:

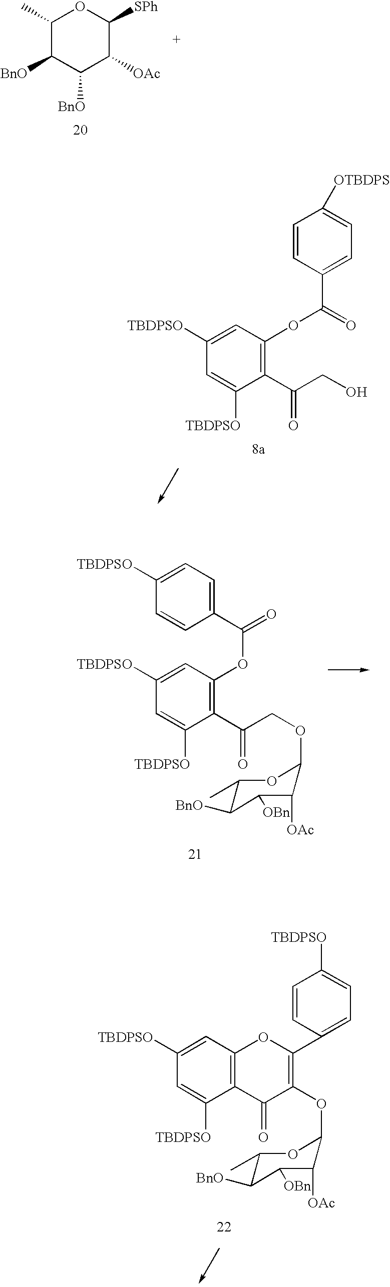

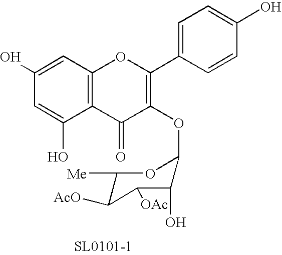

The coupling of 20 (0.1 g) with 8a (1.5 equiv) using O-glycosidation conditions [1-benzenesulfinyl piperidine (1 equiv), tri-t-butylpyrimidine (2 equiv), triflic anhydride (trifluoromethanesulfonic acid anhydride) (1.1 equiv), CH₂Cl₂, −60° C., 1 h] gave 21 (0.1 g, 35%), which was characterized by ¹H NMR. Dehydration of 21 (0.1 g) using K₂CO₃ in pyridine at reflux to get 22 is in progress and the remaining steps from 21 to produce SL0101-1 are well known to those skilled in the art.

1-12. (canceled)

13. A method of specifically inhibiting Rsk activity, said method comprising the step of contacting a Rsk enzyme with a compound represented by the general structure: 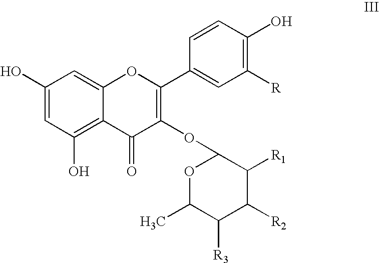
wherein R is H or OH, and R₁, R₂ and R₃ are independently selected from the group consisting of hydroxy —OCOR₄, —COR₄ and C₁-C₄ alkoxy; and
R₄ is H or C₁-C₄ alkyl.

14. The method of claim 13, wherein R is H or OH and R₁, R₂ and R₃ are independently selected from the group consisting of hydroxy and —OCOCH₃.

15. The method of claims 13 or 14 wherein R is H.

16. The method of claims 13, 14 or 15, wherein R₃ is —OCOCH₃.

17. A method of screening for Rsk inhibitory compounds, said method comprising the steps of
contacting a Rsk substrate with Rsk and a potential inhibitory compound under conditions that are permissive for kinase activity;
incubating the substrate for a predetermined length of time; and
determining if the substrate is phosphorylated, wherein a decrease in kinase activity relative to a reaction run in the absence of said potential inhibitory compound identifies an inhibitory compound.

18. The method of claim 17 wherein the step of determining if the substrate is phosphorylated, comprises quantitating the level of phosphorylation through the use of phosphospecific antibodies.

19-47. (canceled)
